(19) United States
(12) Patent Application Publication
(10) Pub. No.: US 2026/0258153 A1
(43) Pub. Date: Sep. 3, 2026

(54) DNA-ENCODED BISPECIFIC ANTIBODIES TARGETING CARBONIC ANHYDRASE 9 AND METHODS OF USE IN CANCER THERAPEUTICS

(71) Applicant: The Wistar Institute of Anatomy and Biology, Philadelphia, PA (US)

(72) Inventors: David Weiner, Merion, PA (US); Ryan O'Connell, Philadelphia, PA (US)

(21) Appl. No.: 18/869,634

(22) PCT Filed: Jun. 1, 2023
(86) PCT No.: PCT/US23/67765

§ 371 Date: Nov. 26, 2024

Related U.S. Application Data
(60) Provisional application No. 63/347,670, filed on Jun. 1, 2022.

(51) Int. Cl. C07K 16/40

(52) U.S. Cl. CPC C07K 16/40; C07K 2317/31

(57) ABSTRACT

Disclosed herein are compositions comprising a recombinant nucleic acid sequence encoding a bispecific therapeutic antibody targeting CA9, a fragment thereof, a variant thereof, or a combination thereof, and methods of use for treating cancer.

### CROSS REFERENCE TO RELATED APPLICATIONS

This application claims priority to U.S. Provisional Application No. 63/347,670, filed Jun. 1, 2022 which is hereby incorporated by reference herein in its entirety.

### TECHNICAL FIELD

The present invention relates to compositions comprising a recombinant nucleic acid sequence for generating one or more synthetic DNA encoded bispecific antibody (DBTE), and functional fragments thereof, in vivo, and methods of preventing and/or treating cancer in a subject by administering said compositions.

### BACKGROUND

Renal cell carcinoma (RCC) is responsible for 90% of kidney cancers and stands as the 9^{th} most common cancer in men and 14^{th} most common cancer in women worldwide. New immune therapies could make a meaningful impact in RCC. DNA-encoded bispecific T cell engagers (DBTEs) are a promising antibody therapies equipped with dual specificity for cancer and T cell antigens, empowering them to direct and stimulate T-cell cytotoxic activity at the tumor site following DNA expression and secretion. The cancer antigen carbonic anhydrase 9 (CA9) is overexpressed in 90% of RCC cases, providing an ideal target for new DBTE therapies.

Bispecific T cell engagers are bispecific antibodies comprised of two single chain variable fragments (scFvs) that can simultaneously bind to two different antigens and bring the cells displaying each individual antigen close to each other. Typically, one arm of the BTE binds to a Tumor Associated Antigen (TAA) and the other end binds to the CD3 epsilon chain on T cells. Engagement of both arms of BTE triggers T cell activation leading to cytolysis of tumor cells. One such BTE targeting CD19 has received FDA approval for treatment of acute lymphoblastic leukemia in 2014 (Jen et al., 2019, Clinical Cancer Research; 25(2):473-7). CD3 based bispecific antibodies can lead to Cytokine Release Syndrome (CRS), which is a major clinical concern (Teachy et al., 2013, Blood, 121(26):5154-7). Additional challenges for treatment with bispecific antibodies include manufacturing limitations and short in vivo half-life resulting in the need for continuous infusions over several weeks and associated increased costs (Zhu et al., 2016, Clinical Pharmacokinetics, 55(10):1271-88). A delivery method resulting in longer in vivo expression could significantly improve the availability of this technology to larger populations.

Previously an approach of using synthetic DNA (synDNA) encoded monoclonal antibodies for immunotherapy with improved expression kinetics has been described (Teachy et al., 2013, Blood, 121(26):5154-7; Zhu et al., 2016, Clinical Pharmacokinetics, 55(10):1271-88; (Perales-Puchalt et al., 2019, Journal of Clinical Investigation Insight, 4(8):e126086)). It is important to develop new T cell redirecting therapies because of their exceptional patient potential; a simplified production scheme which would improve the patient experience, while improving tumor control in vivo. In this regard, a DNA launched Her2 targeting bispecific antibody that was expressed in mice showed impactful tumor control in an animal model of ovarian cancer (Perales-Puchalt et al., 2019, Journal of Clinical Investigation Insight, 4(8):e126086).

Thus there is need in the art for the development of a highly focused CA9 targeting immunotherapy with high potency. The current invention satisfies this need.

### BRIEF SUMMARY OF THE INVENTION

In one embodiment, the invention relates to a nucleic acid molecule encoding one or more synthetic DNA encoded bispecific therapeutic antibodies, or a binding fragment thereof, wherein the one or more synthetic DNA encoded bispecific immune cell engager comprises at least one least one antigen binding domain specific for binding to carbonic anhydrase 9 (CA9), and at least one immune cell engaging domain.

In one embodiment, the immune cell engaging domain targets a T cell, an antigen presenting cell, a natural killer (NK) cell, a neutrophil or a macrophage. In one embodiment, the immune cell engaging domain targets CD3, the T cell receptor (TCR), CD28, CD16, NKG2D, Ox40, 4-1BB, CD2, CD5, CD40, FcgRs, FceRs, FcaRs or CD95. In one embodiment, the immune cell engaging domain targets CD3.

In one embodiment, the nucleic acid molecule comprises a nucleotide sequence encoding an amino acid sequence having at least about 90% identity over an entire length of the amino acid sequence to SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28. In one embodiment, the nucleic acid molecule comprises a binding fragment of an amino acid sequence having at least about 90% identity over at least 65% of the amino acid sequence to SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28. In one embodiment, the nucleic acid molecule comprises an amino acid sequence of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28. In one embodiment, the nucleic acid molecule comprises a binding fragment of an amino acid sequence comprising at least 65% of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28.

In one embodiment, the nucleic acid molecule comprises the six CDR sequences of the amino acid sequences of SEQ ID NO:2 and SEQ ID NO:4. In one embodiment, the binding fragment comprises the twelve CDR sequences of the amino acid sequences of SEQ ID NO:2, SEQ ID NO:4, SEQ ID NO:6, and SEQ ID NO:8.

In one embodiment, the nucleic acid molecule comprises a nucleotide sequence having at least about 90% identity over an entire length of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27. In one embodiment, the nucleic acid molecule comprises a fragment of a nucleotide sequence having at least about 90% identity over at least 65% of the nucleic acid sequence of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27. In one embodiment, the nucleic acid molecule comprises a nucleotide sequence of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27. In one embodiment, the nucleic acid molecule comprises a fragment of a nucleotide sequence comprising at least 65% of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27.

In one embodiment, the fragment encodes the six CDR sequences of the amino acid sequences of SEQ ID NO:2 and SEQ ID NO:4. In one embodiment, the fragment encodes the twelve CDR sequences of the amino acid sequences of SEQ ID NO:2, SEQ ID NO:4, SEQ ID NO:6, and SEQ ID NO:8.

In one embodiment, the nucleotide sequence is operably linked to a nucleic acid sequence encoding an IgE leader sequence.

In one embodiment, the nucleic acid molecule comprises an expression vector.

In one embodiment, the invention relates to a composition comprising at least one nucleic acid molecule encoding one or more synthetic DNA encoded bispecific therapeutic antibodies, or a binding fragment thereof, wherein the one or more synthetic DNA encoded bispecific immune cell engager comprises at least one least one antigen binding domain specific for binding to carbonic anhydrase 9 (CA9), and at least one immune cell engaging domain. In one embodiment, the composition further comprises a pharmaceutically acceptable excipient.

In one embodiment, the invention relates to a method of preventing or treating a disease or disorder in a subject, the method comprising administering to the subject a nucleic acid molecule encoding one or more synthetic DNA encoded bispecific therapeutic antibodies, or a binding fragment thereof, wherein the one or more synthetic DNA encoded bispecific immune cell engager comprises at least one least one antigen binding domain specific for binding to carbonic anhydrase 9 (CA9), and at least one immune cell engaging domain or a composition comprising at least one nucleic acid molecule encoding one or more synthetic DNA encoded bispecific therapeutic antibodies, or a binding fragment thereof, wherein the one or more synthetic DNA encoded bispecific immune cell engager comprises at least one least one antigen binding domain specific for binding to carbonic anhydrase 9 (CA9), and at least one immune cell engaging domain.

In one embodiment, the disease is a benign tumor, cancer or a cancer-associated disease. In one embodiment, the disease is a cancer associated with CA9 expression. In one embodiment, the disease is renal cell carcinoma or hepatocellular carcinoma.

### BRIEF DESCRIPTION OF THE DRAWINGS

FIG. 1A through FIG. 1C: Antibody design, synthesis, and expression. FIG. 1A) Panel of BTE designs that includes the canonical BTE, HLE BTE, and HLV+ BTE formats. FIG. 1B) Plasmid map containing codon-optimized BTEs in pVax1 expression vectors. FIG. 1C) Western blot data confirm expression of the BTE, HLE BTE, and HLV+ BTE with bands corresponding to appropriate molecular weights.

FIG. 2A through FIG. 2H: HLV+ BTE displays highest avidity for target cells. FIG. 2A) Flow cytometry binding curves to ACHN-CA9 (n=3), 293T-CA9 (n=4), and T cells (n=3). FIG. 2B) KD calculations from respective cell binding curves. FIG. 2C) Representative shifts for target cell binding from respective flow cytometry experiments. Secondary antibody controls are reflected in grey. FIG. 2D) z-Movi workflow depicting the insertion of adhered cancer cells, suspended T cells, and compounds to a flow chamber, wherein ultrasound waves pull T cells from cancer cells towards acoustic nodes. The force required to separate cells is recorded at single cell resolution, and the detachment force required is a reflection of the synaptic strength, or avidity, generated by the bispecific antibodies. FIG. 2E) Curves represent the percent of cells remaining bound with increasing detachment force over time. Antibodies were analyzed at 30 nM (n=5), 3 nM (n=3), and 300 μM (n=3). FIG. 2F) Area under curve (AUC) analyses of adjacent force curves at 30 nM (n=5), 3 nM (n=3), and 300 μM (n=3).

FIG. 2G) Single cell resolution of the force required to detach T cells from cancer cells for individual antibodies for respective antibody concentrations. Points represent individual T cells and data represent all replications collectively. FIG. 2H) Representative images of the flow chamber at 1000 pN display localization of detached T cells at acoustic nodes for lower avidity antibodies at 30 nM.

FIG. 3A through FIG. 3E. HLV+ BTE activates T cells with the highest potency. FIG. 3A) CD69+ expression on CD4+ and CD8+ T cells after 24 hours of co-incubation with antibodies and SKRC-52 cells (n=5). FIG. 3B) CD25+ expression on CD4+ and CD8+ T cells after 24 hours of co-incubation with antibodies and SKRC-52 cells (n=5). FIG. 3C) CD69+ expression on CD4+ and CD8+ T cells after 24 hours of co-incubation with antibodies and 293T-CA9 cells (n=4). FIG. 3D) CD25+ expression on CD4+ and CD8+ T cells after 24 hours of co-incubation with antibodies and 293T-CA9 cells (n=4). FIG. 3E) IL-2, TNF-α, sFASL, IL-17A, and IFN-γ cytokine concentrations in the supernatant of co-incubated SKRC-52 cells, T cells, and antibodies (10 nM or 1 μM) after 24 hours (n=4). Irrelevant antibody control quantifications were BQL for IL-2, TNF-α, and IFN-γ from 10 nM treatment. IL-2, sFasL, and IL-17A concentrations were BQL from 1 μM treatments.

FIG. 4A through FIG. 4C: HLV+ BTE induces the T cell-mediated cytotoxicity of target cells with the highest potency. FIG. 4A) The percent of terminal cytotoxicity at increasing concentrations of antibody treatment for the following target cell lines: SKRC-52 (n=4), A-498 (n=3), 786-O (n=2), ACHN-CA9 (n=3), and 293T-CA9 (n=2). Percent cytotoxicity was calculated as (1-(Treatment/Target cell control))*100) at the terminal experimental timepoint. FIG. 4B) Representative real-time analyses of cell growth over time for 10 nM and 1 μM antibody treatments for the adjacent cell lines illustrate potent cytotoxicity. FIG. 4C) Representative images taken at the terminal timepoint of single experiments for each treatment group at 10 nM, illustrating the cytotoxicity of the adjacent target cell lines.

FIG. 5A through FIG. 5G: HLV+ BTE displays long half-life and improved tumor distribution. FIG. 5A) Balb/c mice (n=3) were administered a single 2.5 mg/kg (IV) dose of antibodies in PBS and circulating antibody was quantified from sera via His-capture ELISA for pharmacokinetic analysis. Serum antibody concentrations are displayed in both μm/mL and uM units. Pharmacokinetic calculations pertaining to these data are in Table 1. FIG. 5B) The tumor distribution PK analysis workflow begun with an ACHN-CA9 xenograft in NSG mice (n=4) and treatments were administered with a single 0.5 mg/kg (IV) dose in PBS when tumors reached 200 mm3. Tumors and serum were harvested after 24 hours, tumors were homogenized, and antibody concentrations were analyzed by His-capture ELISA. FIG. 5C) A tumor distribution summary plot of intratumoral concentrations against serum, with the resulting tumor/serum concentration ratio (×10⁻³) represented by point size. FIG. 5D-5E) Serum (FIG. 5D) and intratumoral (FIG. 5E) antibody concentrations at 24 hours (n=4). Intratumoral antibody concentrations are given in both ng/mL and pM units. FIG. 5F) The comparative distribution of antibodies into the tumor compartment is considered as the tumor/serum, ng/ml concentration ratio (×10⁻³). This analysis is plotted with or without the traditional BTE. FIG. 5G) Tumor volume over time for individual tumors and the mean for each treatment. FIG. 5H) Tumor volume on days 25 and 30.

FIG. 6A through FIG. 6E: DNA-delivered HLV+ BTE demonstrates the most durable activity. FIG. 6A) DNA-based BTEs are administered as plasmid DNA with hyaluronidase (50 U/mouse) and electroporation for enhanced cellular uptake. Myocytes synthesize antibodies in situ and secrete into circulation. In tumor models, donor T cells are administered (IP) and bispecific antibody efficacy is assessed. Serum can be collected from antibody-treated mice, whether naïve or from tumor models, for in vitro functional assessments of the in vivo-synthesized antibodies. FIG. 6B) Naïve, balb/c mice were administered 200 μg of BTE, HLE, or HLV+ BTE DNA (n=5). Sera collected over time were used to stain ACHN and ACHN-CA9 cells by flow cytometry. ACHN cells are represented in grey while colored shifts represent ACHN-CA9 cells up to day 21. FIG. 6C) DBTE sera were also used to stain donor T cells by flow cytometry from days 0-21. FIG. 6D) DBTE sera from the balb/c mice (n=5) were tested for their ability to induce cytotoxicity of SKRC-52 cells in vitro, using an xCELLigence system. Diminished cell indices represent target cell death. Negative controls included sera from empty pVax1-treated mice, while recombinant BTE (10 nM) was used as a positive control. FIG. 6E) Tumor volume over time for individual tumors and the mean for each treatment.

FIG. 7 demonstrates the Flow Cytometry gating strategy and results.

FIG. 8 demonstrates the results of serial dilutions.

FIG. 9A through FIG. 9C: BTEs do not induce off-target toxicity of CA9 non-expressing cells. FIG. 9A) xCELLigence real-time analysis workflow involves the co-incubation of target cells, donor T cells, and antibody treatments. A current moves through each well base, and impedance measurements in arbitrary cell index units reflect adherent cell density. Increasing cell index reflects cell proliferation, and reduced cell index is an indirect measurement of cytotoxicity whereupon dying cells dissociate from the well base and relieve impedance. The in-unit camera offers well photos to visualize cell behavior over time. FIG. 9B) Real-time cytotoxicity analyses from donor T cells and antibody treatments (10 nM) of the non-CA9 expressing cell lines ACHN, OVCAR3, DAOY, and 293T (n=1 per cell line). Data are represented as the difference from no-treatment control, where this control's cell index is subtracted from treatment groups. No change from baseline reflects no cytotoxicity. FIG. 9C) Representative images taken at the terminal, 24-hour timepoint of the preceding experiments show no cytotoxicity of ACHN, OVCAR3, DAOY, and 293T cells from antibody treatment (10 nM), compared to the T cells only group.

FIG. 10 depicts experimental results demonstrating that administration of BTE reduces tumor volume.

### DETAILED DESCRIPTION

The present invention relates to compositions comprising a recombinant nucleic acid sequence encoding a bispecific T cell engaging antibody (DBTE) targeting CA9, a fragment thereof, a variant thereof, or a combination thereof. In one embodiment, the DBTE of the invention is an immune cell engaging therapeutic antibody. The composition can be administered to a subject in need thereof to facilitate in vivo expression and formation of a DBTE.

In one embodiment, the CA9 DBTE comprisies at least one antigen binding domain specific for binding to CA9, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In various embodiments, the antigen binding domain comprises a nucleotide sequence encoding an antibody, a fragment thereof, or a variant thereof specific for binding to CA9. In one embodiment, the antibody or fragment thereof is a DNA encoded monoclonal antibody (DMAb) or a fragment or variant thereof.

In one embodiment, the bispecific DBTE is specific for binding CA9, and recruiting a T cell to a cell expressing CA9. In some embodiments, cells expressing CA9 are cancer cells. In some embodiments, cells expressing CA9 are renal cell carcinoma or hepatocellular carcinoma cells. Therefore, in one embodiment, the invention provides compositions comprising one or more bispecific CA9 DBTE and methods for use in treating or preventing cancer (e.g., renal cell carcinoma or hepatocellular carcinoma), or a disease or disorder associated with cancer in a subject.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering an antibody of the present invention to a subject prior to onset of the disease. Suppressing the disease involves administering a antibody of the present invention to a subject after induction of the disease but before its clinical appearance. Repressing the disease involves administering an antibody of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Compositions

In one embodiment, the present invention relates to compositions comprising a recombinant nucleic acid sequence encoding a bispecific therapeutic antibody (DBTE) comprising an antigen binding domain specific for binding CA9, a fragment thereof, a variant thereof, or a combination thereof. The compositions, when administered to a subject in need thereof, can result in the generation of a synthetic bispecfic CA9 antibody in the subject.

In one embodiment, the bispecific CA9 DBTE comprisies at least one antigen binding domain specific for binding to CA9, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In various embodiments, the antigen binding domain comprises an antibody, a fragment thereof, or a variant thereof specific for binding to CA9.

In one embodiment, a nucleotide sequence encoding a CA9 DBTE encodes at least one amino acid sequence of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28 or a fragment or variant thereof. In one embodiment, the fragment of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28 is a binding fragment comprising at least one, at least two, at least three, at least four, at least five, at least six, at least seven, at least 8, at least 9, at least 10, at least 11 or all 12 CDR sequences of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28. In some embodiments, the binding fragment comprises at least three CDR sequences of SEQ ID NO:2 or SEQ ID NO:4. In some embodiments, the binding fragment comprises at least six CDR sequences of SEQ ID NO:2 and SEQ ID NO:4.

In one embodiment, a nucleotide sequence encoding a CA9 DBTE comprises at least one nucleotide sequence of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27 or a fragment or variant thereof. In one embodiment, the fragment of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27 encodes a binding fragment of a DBTE of the invention, and comprises at least one, at least two, at least three, at least four, at least five, at least six, at least seven, at least eight, at least nine, at least ten, at least eleven or all twelve CDR coding sequences of SEQ ID NO:1, SEQ ID NO:3, SEQ ID NO:5 and SEQ ID NO:7, encoding at least one, at least two, at least three, at least four, at least five, at least six, at least seven, at least eight, at least nine, at least ten, at least eleven or all twelve CDR sequences of SEQ ID NO:2, SEQ ID NO:4, SEQ ID NO:6 and SEQ ID NO:8. In some embodiments, the fragment of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27 encodes a binding fragment of a DBTE of the invention comprising at least three CDR sequences of SEQ ID NO:2 or SEQ ID NO:4. In some embodiments, the fragment of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27 encodes a binding fragment of a DBTE of the invention comprising at least six CDR sequences of SEQ ID NO:2 and SEQ ID NO:4.

In certain embodiments, the composition can treat, prevent, and or/protect against a disease or disorder associated with expression of CA9. In one embodiment, the composition of the invention can treat, prevent and/or protect against any disease, disorder, or condition associated with expression of a CA9. In certain embodiments, the composition can treat, prevent, and or/protect against cancer. In one embodiment, the composition can treat, prevent, and or/protect against renal cell carcinoma or hepatocellular carcinoma.

The synthetic antibody (e.g., DBTE) can treat, prevent, and/or protect against disease in the subject administered the composition. The synthetic antibody (e.g., DBTE) can promote survival of the disease in the subject administered the composition. The synthetic antibody (e.g., DBTE) can provide at least about 50%, 55%, 60%, 65%, 70%, 75%, 80%, 85%, 90%, 95%, or 100% survival of the disease in the subject administered the composition. In other embodiments, the synthetic antibody (e.g., DBTE) can provide at least about 65%, 66%, 67%, 68%, 69%, 70%, 71%, 72%, 73%, 74%, 75%, 76%, 77%, 78%, 79%, or 80% survival of the disease in the subject administered the composition.

The composition can result in the generation of the synthetic antibody (e.g., DBTE) in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody (e.g., DBTE) in the subject within at least about 1 day, 2 days, 3 days, 4 days, 5 days, 6 days, 7 days, 8 days, 9 days, or 10 days of administration of the composition to the subject. The composition can result in generation of the synthetic antibody (e.g., DBTE) in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody (e.g., DBTE) in the subject more quickly than the generation of an endogenous antibody in a subject who is administered an antigen to induce a humoral immune response. The composition can result in the generation of the synthetic antibody (e.g., DBTE) at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

### Recombinant Nucleic Acid Sequence

As described above, the composition can comprise a recombinant nucleic acid sequence. The recombinant nucleic acid sequence can encode the synthetic antibody (e.g., DBTE), a fragment thereof, a variant thereof, or a combination thereof. The antibody is described in more detail below.

The recombinant nucleic acid sequence can be a heterologous nucleic acid sequence. The recombinant nucleic acid sequence can include at least one heterologous nucleic acid sequence or one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### Heavy Chain Polypeptide

The recombinant nucleic acid sequence construct can include a heterologous nucleic acid encoding a heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### Light Chain Polypeptide

The recombinant nucleic acid sequence construct can include a heterologous nucleic acid sequence encoding a light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides. In one embodiment, the linker sequence is a G4S linker sequence, having an amino acid sequence of GGGGSGGGGSGGGGS (SEQ ID NO:29).

### Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody (e.g., DBTE). In particular, the heavy chain polypeptide and the light chain polypeptide can interact with one another such that assembly results in the synthetic antibody (e.g., DBTE) being capable of binding the antigen. In other embodiments, the heavy chain polypeptide and the light chain polypeptide can interact with one another such that assembly results in the synthetic antibody (e.g., DBTE) being more immunogenic as compared to an antibody not assembled as described herein. In still other embodiments, the heavy chain polypeptide and the light chain polypeptide can interact with one another such that assembly results in the synthetic antibody (e.g., DBTE) being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### RNA

In one embodiment, the nucleic acid is an RNA molecule. In one embodiment, the RNA molecule is transcribed from a DNA sequence. Accordingly, in one embodiment, the invention provides an RNA molecule encoding one or more of the synthetic antibodies of the invention. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly (A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### Antibody

In some embodiments, the invention relates to a recombinant nucleic acid sequence encoding an antibody, a fragment thereof, a variant thereof, or a combination thereof. The antibody can bind or react with an antigen, which is described in more detail below. In some embodiments, the antibody is a DNA encoded monoclonal antibody (DMAb), a fragment thereof, or a variant thereof. In some embodiments the fragment is an ScFv fragment. In some embodiments, the antibody is a DNA encoded bispecific T cell engagers (BiTE), a fragment thereof, or a variant thereof.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CHI region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

### ScFv Antibody

In one embodiment, the DBTE of the invention is a ScFv DBTE. In one embodiment, ScFv DBTE relates to a Fab fragment without the of CHI and CL regions. Thus, in one embodiment, the ScFv DBTE relates to a Fab fragment DBTE comprising the VH and VL. In one embodiment, the ScFv DBTE comprises a linker between VH and VL. In one embodiment, the ScFv DBTE is an ScFv-Fc DBTE. In one embodiment, the ScFv-Fc DBTE comprises the VH, VL and the CH2 and CH3 regions. In one embodiment, the ScFv-Fc DBTE comprises a linker between VH and VL. In one embodiment, the ScFv DBTE of the invention has modified expression, stability, half-life, antigen binding, heavy chain—light chain pairing, tissue penetration or a combination thereof as compared to a parental DBTE.

In one embodiment, the ScFv DBTE of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DBTE.

In one embodiment, the ScFv DBTE of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DBTE.

In one embodiment, the ScFv DBTE of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DBTE.

In one embodiment, the ScFv DBTE of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DBTE.

In one embodiment, the ScFv DBTE of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DBTE.

In one embodiment, the ScFv DBTE of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain—light chain pairing than the parental DBTE.

In one embodiment, the anti-HER2 scFv antibody comprises an amino acid sequence at least 90% homologous to the amino acid sequence of SEQ ID NO: 66, or a fragment of an amino acid sequence at least 90% homologous to the amino acid sequence of SEQ ID NO: 66. In one embodiment, the anti-HER2 scFv antibody comprises the amino acid of SEQ ID NO: 66, or a fragment of the amino acid sequence of SEQ ID NO: 66. In one embodiment, the anti-HER2 scFv antibody comprises an amino acid sequence at least 90% homologous to the amino acid sequence encoded by SEQ ID NO: 65, or a fragment of an amino acid sequence at least 90% homologous to the amino acid sequence encoded by one of SEQ ID NO: 65. In one embodiment, the anti-HER2 scFv antibody comprises the amino acid sequence encoded by SEQ ID NO: 65, or a fragment of the amino acid sequence encoded by SEQ ID NO: 65.

### Monoclonal Antibodies

In one embodiment, the invention provides anti-HER2 antibodies. The antibodies may be intact monoclonal antibodies, and immunologically active fragments (e.g., a Fab or (Fab)₂ fragment), a monoclonal antibody heavy chain, or a monoclonal antibody light chain.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

In one embodiment, the anti-HER2 antibody is optimized for expression in human. In one embodiment, the anti-HER2 antibody comprises an amino acid sequence at least 90% homologous to the amino acid sequence of SEQ ID NO: 62, or a fragment of an amino acid sequence at least 90% homologous to the amino acid sequence of SEQ ID NO: 62. In one embodiment, the anti-HER2 antibody comprises the amino acid of SEQ ID NO: 62, or a fragment of the amino acid sequence of SEQ ID NO: 62. In one embodiment, the anti-HER2 antibody comprises an amino acid sequence at least 90% homologous to the amino acid sequence encoded by SEQ ID NO: 61, or a fragment of an amino acid sequence at least 90% homologous to the amino acid sequence encoded by one of SEQ ID NO: 61. In one embodiment, the anti-HER2 antibody comprises the amino acid sequence encoded by SEQ ID NO: 61, or a fragment of the amino acid sequence encoded by SEQ ID NO: 61.

In one embodiment, the anti-HER2 antibody is optimized for expression in mouse. In one embodiment, the anti-HER2 antibody comprises an amino acid sequence at least 90% homologous to the amino acid sequence of SEQ ID NO: 64, or a fragment of an amino acid sequence at least 90% homologous to the amino acid sequence of SEQ ID NO: 64. In one embodiment, the anti-HER2 antibody comprises the amino acid of SEQ ID NO: 64, or a fragment of the amino acid sequence of SEQ ID NO: 64. In one embodiment, the anti-HER2 antibody comprises an amino acid sequence at least 90% homologous to the amino acid sequence encoded by SEQ ID NO: 61, or a fragment of an amino acid sequence at least 90% homologous to the amino acid sequence encoded by one of SEQ ID NO: 63. In one embodiment, the anti-HER2 antibody comprises the amino acid sequence encoded by SEQ ID NO: 63, or a fragment of the amino acid sequence encoded by SEQ ID NO: 63.

### Bispecific T Cell Engager

As described above, the recombinant nucleic acid sequence can encode a bispecific T cell engager (BiTE), a fragment thereof, a variant thereof, or a combination thereof. The antigen targeting domain of the BiTE can bind or react with the antigen, which is described in more detail below.

The antigen targeting domain of the BiTE may comprise an antibody, a fragment thereof, a variant thereof, or a combination thereof. The antigen targeting domain of the BiTE may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding domain, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antigen targeting domain of the BiTE can be the Fab or F(ab′)₂. The Fab can include the heavy chain polypeptide and the light chain polypeptide. The heavy chain polypeptide of the Fab can include the VH region and the CHI region. The light chain of the Fab can include the VL region and CL region.

The antigen targeting domain of the BiTE can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antigen targeting domain of the BiTE can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

In one embodiment, at least one of the antigen binding domaing and the immune cell engaging domain of the DBiTE of the invention is a ScFv DNA encoded monoclonal antibody (ScFv DBTE) as described in detail above.

### Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments, one of the binding sites is capable of an tumor antigen. In some embodiments, the binding site included in the Fab fragment is a binding site specific for a tumor antigen. In some embodiments, the binding site included in the single chain Fv fragment is a binding site specific for a tumor antigen such as CD19, BCMA, CD33, FAP, FSHR, EGFR, PSMA, CD123 or Her2.

In some embodiments, one of the binding sites of an antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing.alpha.- and .beta.-chains, in some embodiments, it encompasses .gamma.-chains and .delta.-chains (supra). Accordingly, in some embodiments, the TCR is TCR (alpha/beta) and in some embodiments, it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments, a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the antibody molecule binds a tumor antigen and the second binding site binds a T cell specific receptor molecule and/or a natural killer (NK) cell specific receptor molecule.

In some embodiments, the first binding site of the antibody molecule binds CD19, BCMA, CD33, FAP, FSHR, EGFR, PSMA, CD123 or Her2, and the second binding site binds a T cell specific receptor molecule and/or a natural killer (NK) cell specific receptor molecule. In some embodiments, the first binding site of the antibody molecule binds CD19, BCMA, CD33, FAP, FSHR, EGFR, PSMA, CD123 or Her2 and the second binding site binds one of CD3, TCR, CD28, CD16, NKG2D, Ox40, 4-1BB, CD2, CD5, CD40, FcgRs, FceRs, FcaRs and CD95. In some embodiments, the first binding site of the antibody molecule binds CD19, BCMA, CD33, FAP, FSHR, EGFR, PSMA, CD123 or Her2 and the second binding site binds CD3.

In some embodiments, the first binding site of the antibody molecule binds a T cell specific receptor molecule and/or a natural killer (NK) cell specific receptor molecule and the second binding site binds a tumor antigen. In some embodiments, the first binding site of the antibody binds a T cell specific receptor molecule and/or a natural killer (NK) cell specific receptor molecule and the second binding site binds CD19, BCMA, CD33, FAP, FSHR, EGFR, PSMA, CD123 or Her2. In some embodiments, the first binding site of the antibody binds one of CD3, TCR, CD28, CD16, NKG2D, Ox40, 4-1BB, CD2, CD5, CD40, FcgRs, FceRs, FcaRs and CD95, and the second binding site binds CD19, BCMA, CD33, FAP, FSHR, EGFR, PSMA, CD123 or Her2. In some embodiments, the first binding site of the antibody binds CD3, and the second binding site binds CD19, BCMA, CD33, FAP, FSHR, EGFR, PSMA, CD123 or Her2.

In one embodiment the bispecific antibody of the invention comprises a DBiTE, comprising one or more scFv antibody fragments as described herein, thereby allowing the DBiTE to bind or react with the desired target molecules.

In one embodiment the DBiTE, comprises a nucleic acid molecule encoding a first scFv specific for binding to a target disease-specific antigen linked to a second scFv specific for binding to a T cell specific receptor molecule. The linkage may place the first and second domains in any order, for example, in one embodiment, a nucleotide sequence encoding a scFv specific for binding to a target disease-specific antigen is oriented 5′ (or upstream) to a nucleotide sequence encoding a scFv specific for binding to a T cell specific receptor molecule. In another embodiment, a nucleotide sequence encoding a scFv specific for binding to a target disease-specific antigen is oriented 3′ (or downstream) to a nucleotide sequence encoding a scFv specific for binding to a T cell specific receptor molecule.

### Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### Extension of Antibody Half-Life

As described above, the synthetic antibody (e.g., DBTE, ScFv antibody fragment, DBTE) may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### CAR Molecules

In one embodiment, the invention provides a chimeric antigen receptor (CAR) comprising a binding domain comprising a CA9 antibody of the invention. In one embodiment, the CAR comprises an antigen binding domain. In one embodiment, the antigen binding domain is a targeting domain, wherein the targeting domain directs the T cell expressing the CAR to a CA9 expressing cell. For example, in one embodiment, the targeting domain comprises an antibody, antibody fragment, or peptide that specifically binds to CA9.

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is a CA9 antibody of the invention or a variant thereof, such as an scFV fragment of a CA9 antibody of the invention specific for binding to CA9.

### Antigen

In one embodiment, the synthetic antibody (e.g., DBTE, ScFv antibody fragment, DBTE) is directed to CA9 or a fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

In one embodiment, a synthetic DNA encoded bispecific immune cell engager of the invention targets two or more antigens. In one embodiment, at least one antigen of a bispecific antibody is CA9. In one embodiment, at least one antigen of a bispecific antibody is a T-cell activating antigen.

### T Cell Specific Receptor

In one embodiment, the bispecific DBTE of the invention comprises a scFv of a T cell specific receptor. T cell specific receptors include, but are not limited to, CD3, TCR, CD28, CD16, NKG2D, Ox40, 4-1BB, CD2, CD5, CD40, FcgRs, FceRs, FcaRs and CD95.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Method of Generating the Synthetic Antibody

The present invention also relates a method of generating the synthetic therapeutic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### Method of Delivery of the Composition

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, PA) or Elgen electroporator (Inovio Pharmaceuticals, Plymouth Meeting, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### Methods

In one embodiment, the invention provides methods of administering a synthetic antibody (e.g., bispecific DBTE) to a subject in need thereof. Also provided herein is a method of treating, protecting against, and/or preventing disease in a subject in need thereof by generating the synthetic antibody (e.g., bispecific DBTE) in the subject. The method can include administering the composition to the subject. Administration of the composition to the subject can be done using the method of delivery described above.

In certain embodiments, the invention provides a method of treating protecting against, and/or preventing cancer. In one embodiment, the method treats, protects against, and/or prevents tumor growth. In one embodiment, the method treats, protects against, and/or prevents cancer progression. In one embodiment, the method treats, protects against, and/or prevents cancer metastasis.

In one embodiment, the invention provides methods for preventing growth of benign tumors, such as, but not limited to, uterine fibroids. The methods comprise administering an effective amount of one or more of the compositions of the invention to a subject diagnosed with a benign tumor.

Upon generation of the synthetic antibody (e.g., DBTE) in the subject, the synthetic antibody (e.g., DBTE) can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### Cancer Therapy

The invention provides methods of treating or preventing cancer, or of treating and preventing growth or metastasis of tumors. Related aspects of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective amount of a composition of the invention. The invention further provides a method of inhibiting metastasis in an individual in need thereof, the method comprising administering to the individual an effective metastasis-inhibiting amount of any one of the compositions described herein.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells. As such, the compositions of the invention can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of one or more of the compositions of the invention to a subject diagnosed with a malignant tumor or cancerous cells or to a subject having a tumor or cancerous cells.

The following are non-limiting examples of cancers that can be treated by the methods and compositions of the invention: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor. In one embodiment, the cancer is associated with expression of CA9. In one embodiment, the cancer is renal cell carcinoma or hepatocellular carcinoma.

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### Nanoparticle Formulations

In one embodiment, the immunogenic composition of the invention may comprise a nanoparticle, including but not limited to a lipid nanoparticle (LNP), comprising a CA9 antibody of the invention, or a LNP comprising a nucleic acid encoding a CA9 antibody of the invention. In some embodiments, the composition comprises or encodes all or part of a CA9 binding molecule of the invention, or an immunogenically functional equivalent thereof. In some embodiments, the composition comprises an mRNA molecule that encodes all or part of a CA9 binding molecule of the invention.

In one embodiment, the LNP comprises or encapsulates an RNA molecule encoding at least one amino acid sequence of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28, or a fragment or variant thereof.

In one embodiment, the composition further comprises one or more additional immunostimulatory agents. Immunostimulatory agents include, but are not limited to, an additional antigen or antigen binding molecule, an immunomodulator, or an adjuvant.

### Generation of Synthetic Antibodies In Vitro and Ex Vivo

In one embodiment, the synthetic antibody (e.g., bispecfic DBTE) is generated in vitro or ex vivo. For example, in one embodiment, a nucleic acid encoding a synthetic antibody (e.g., bispecfic DBTE) can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

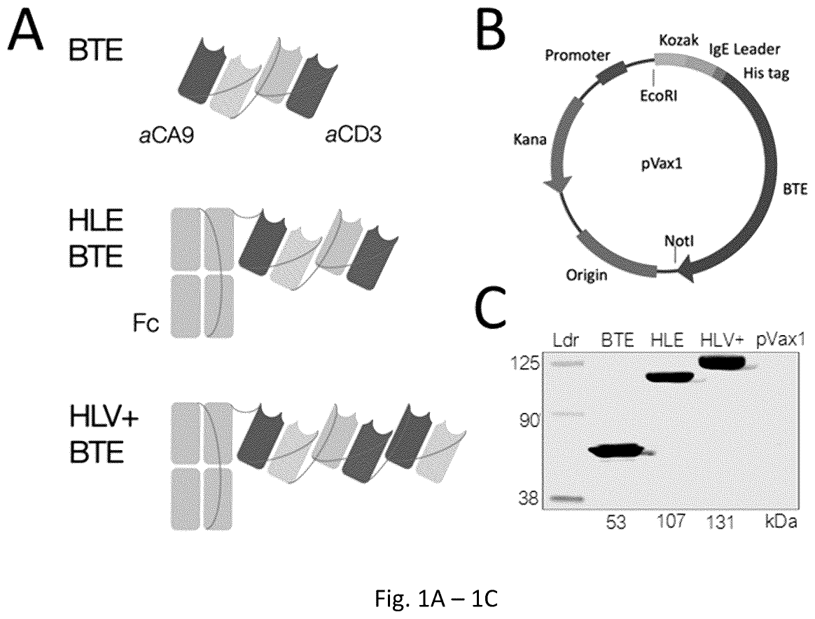

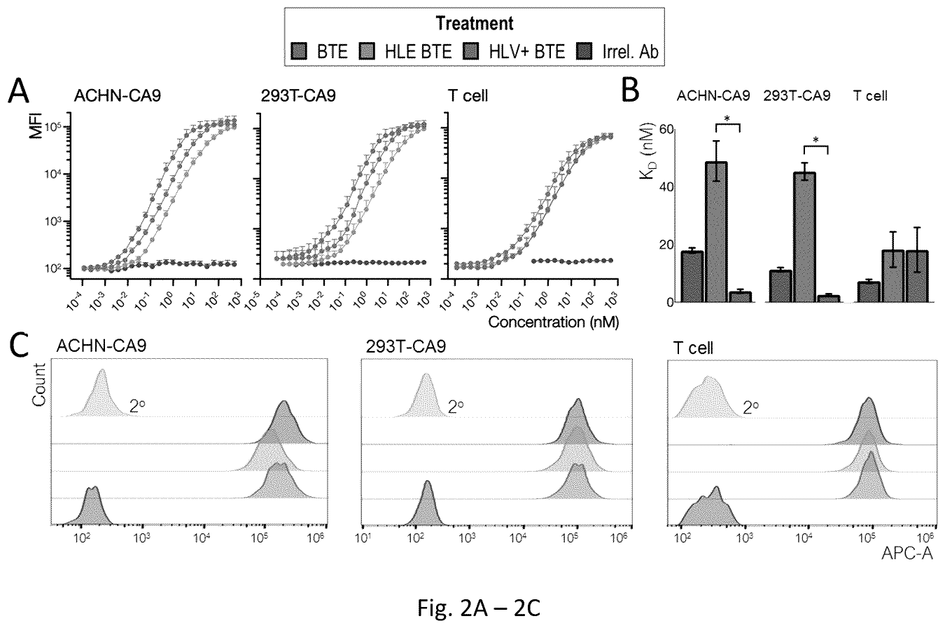

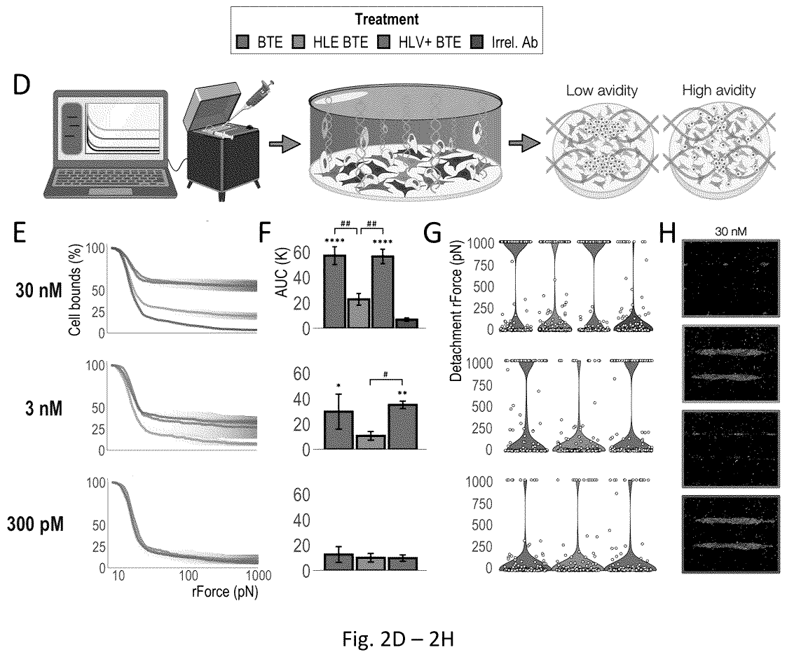

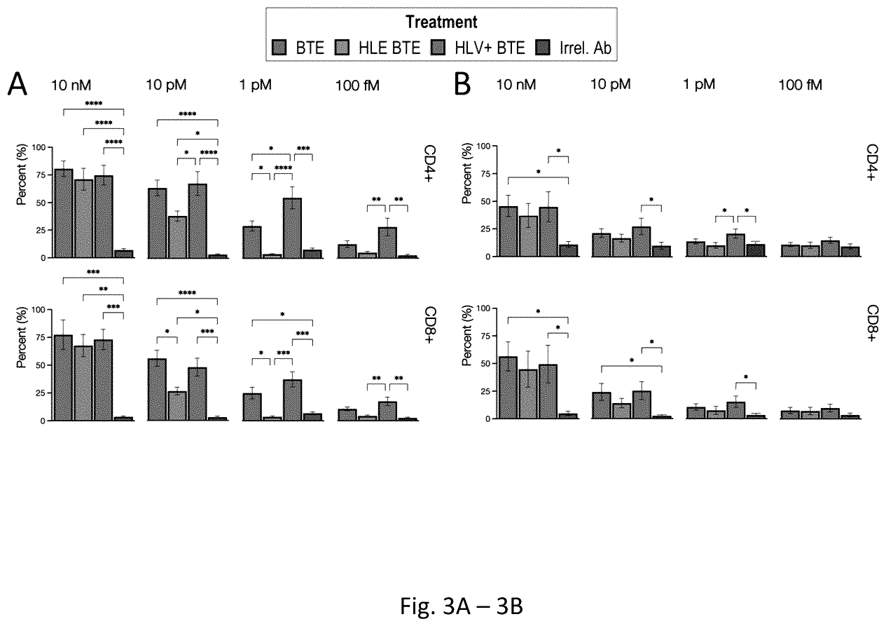

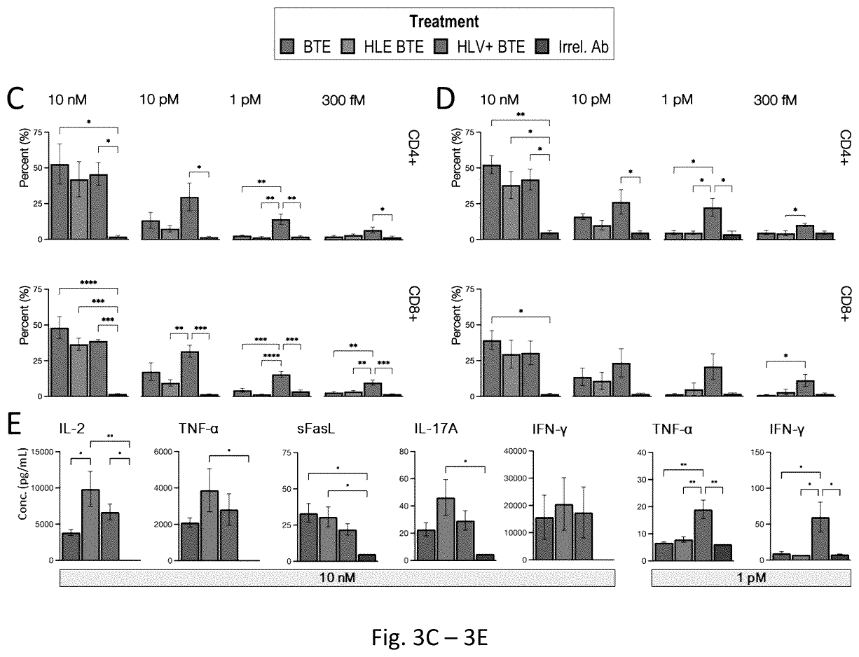

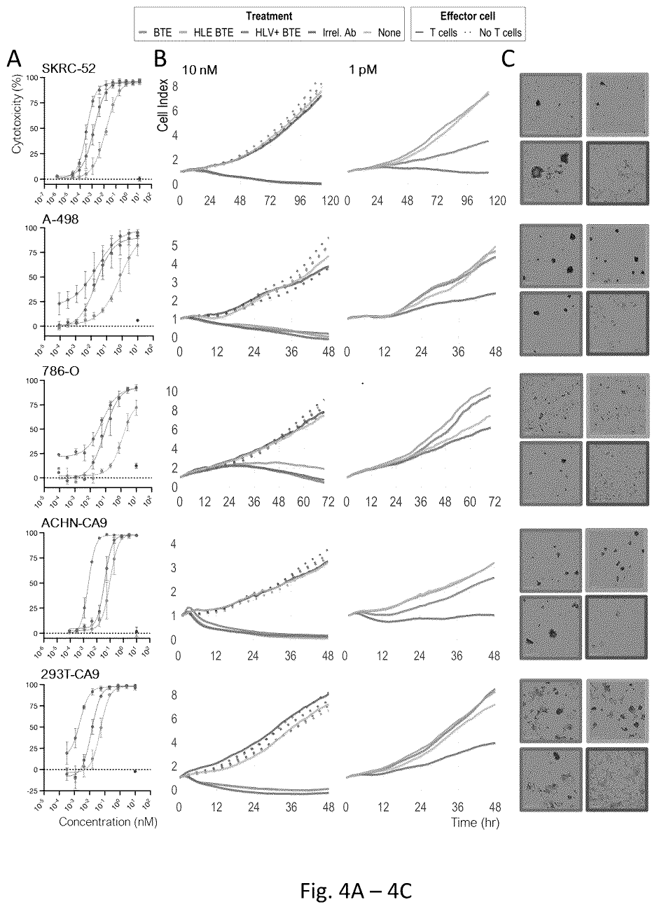

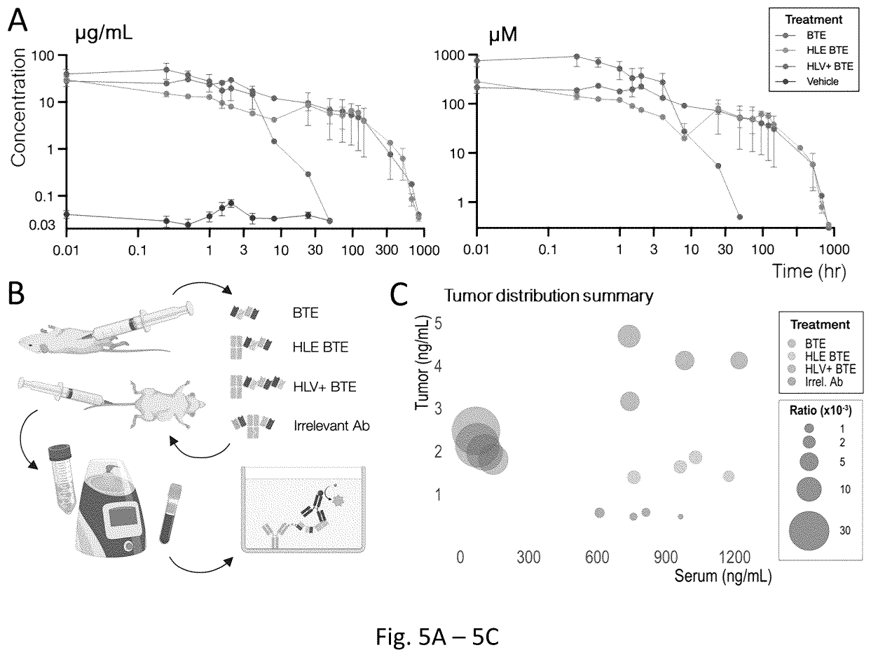

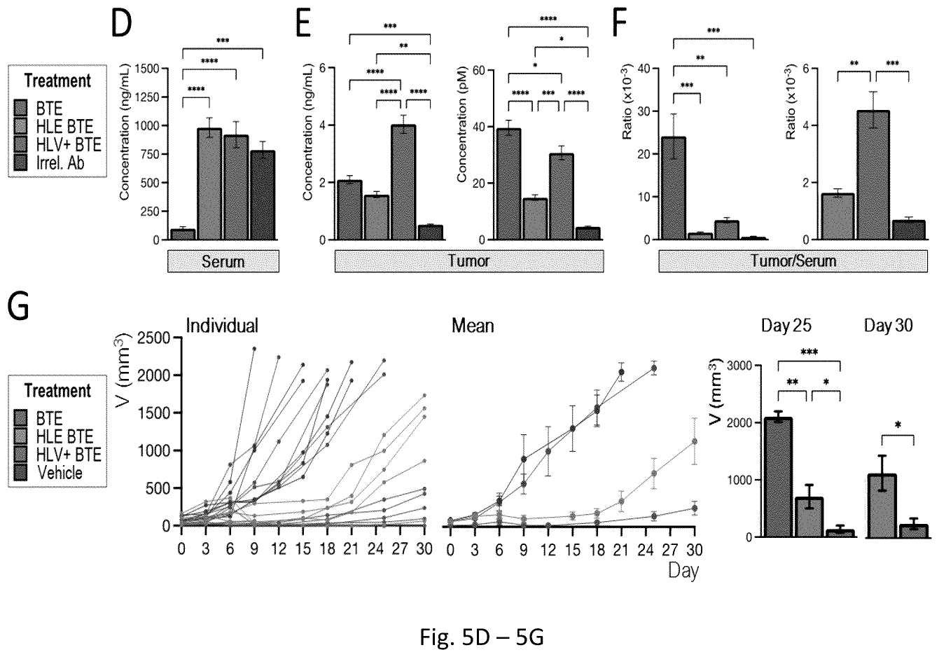

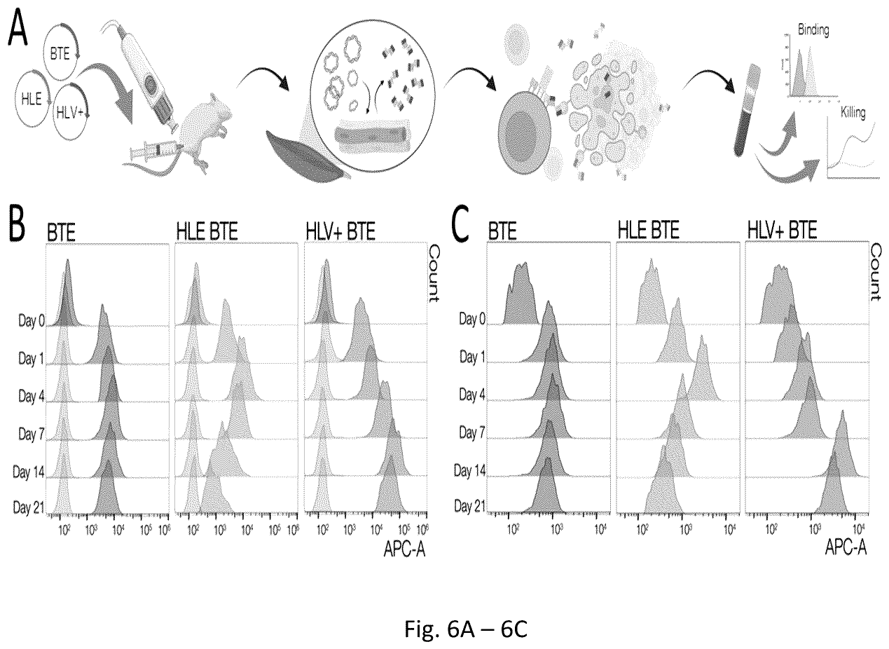

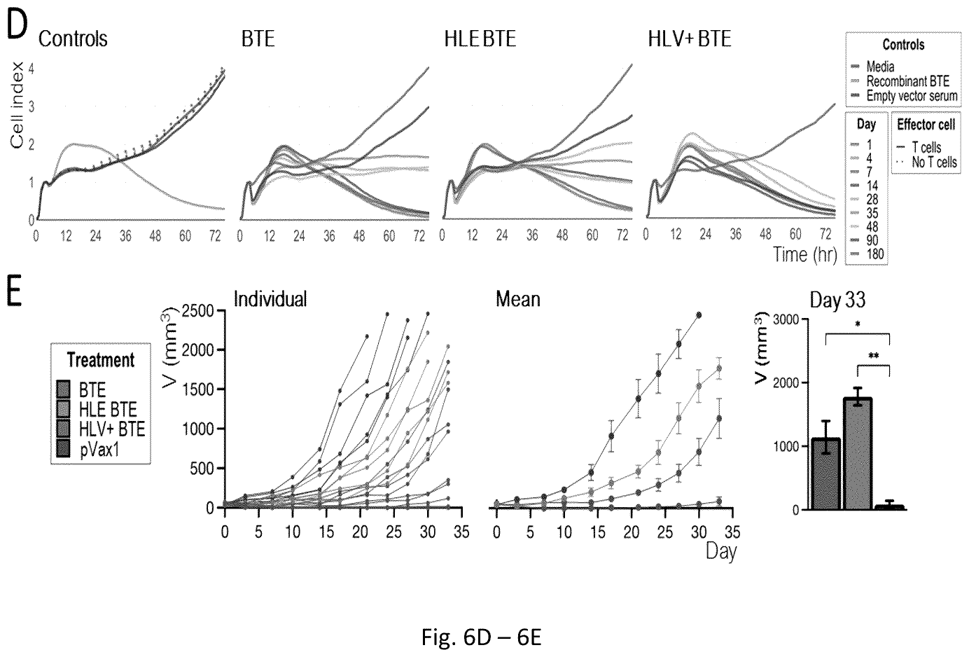

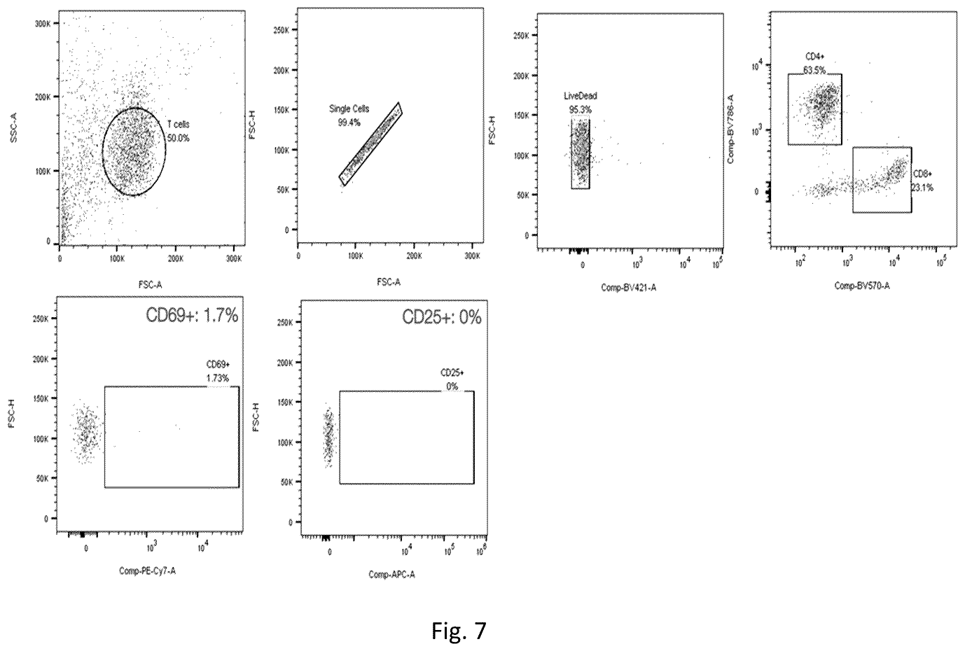

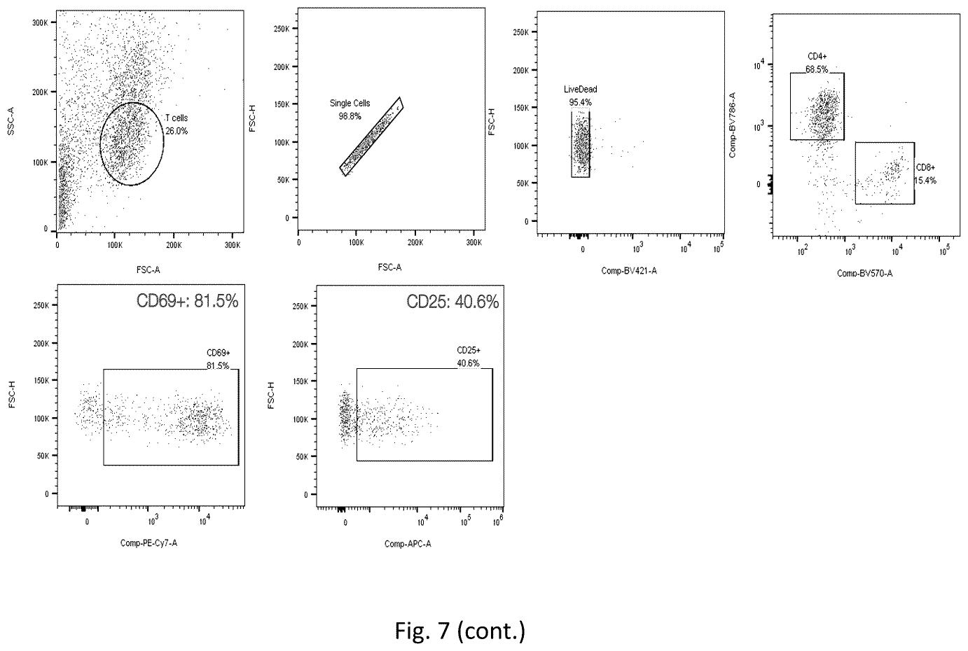

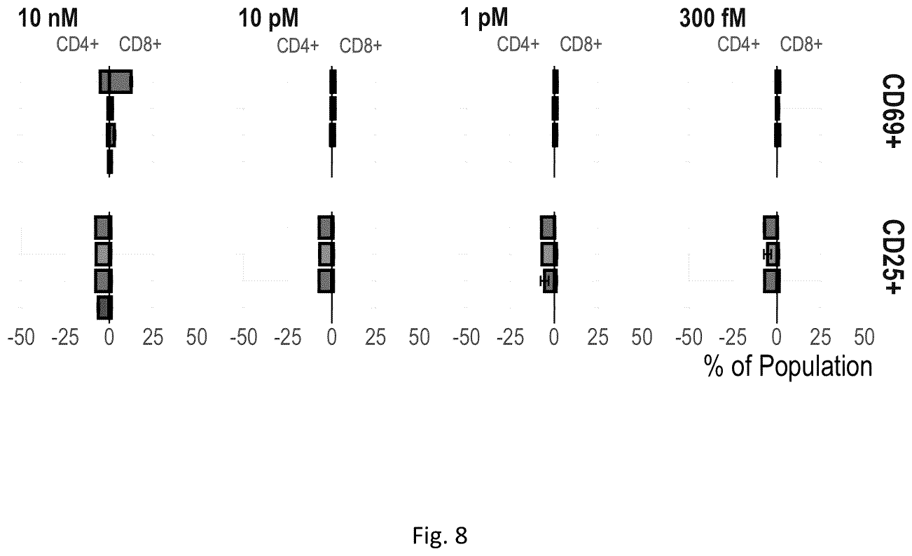

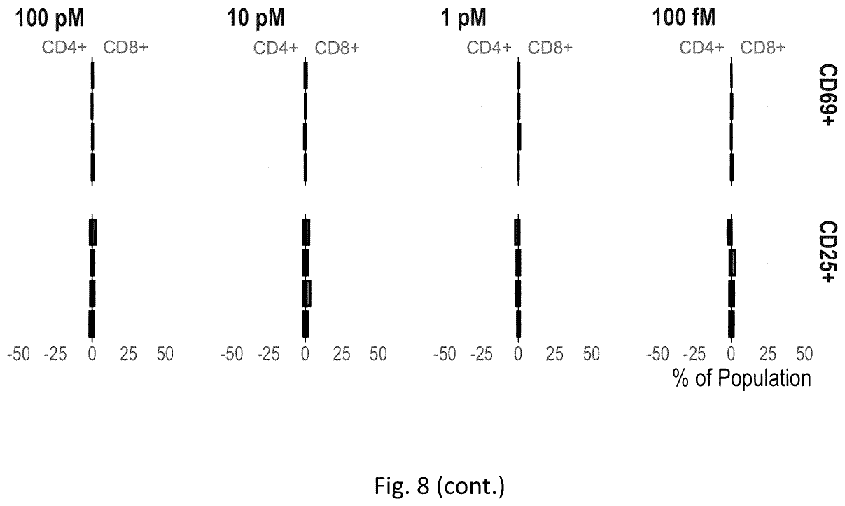

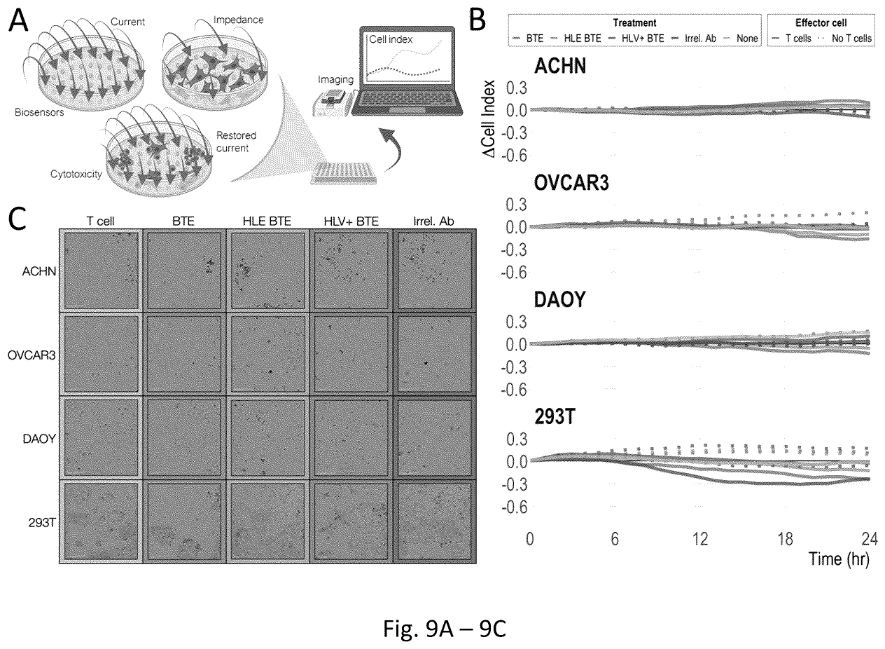

Here a panel of novel CA9-targeted DBTEs was developed that includes canonical and half-life extended DBTEs, as well as a novel half-life/valency-enhanced (HLV) design with bivalency for CA9 (FIG. 1). The data provided demonstrate that the HLV+ BTE displays the highest avidity for target cells (FIG. 2). Further, HLV+ BTE activates T cells with the highest potency (FIG. 3), and HLV+ BTE induces the T cell-mediated cytotoxicity of target cells with the highest potency (FIG. 4). HLV+ BTE displays long half-life and improved tumor distribution (FIG. 5). Further, the data demonstrated that DNA-delivered HLV+ BTE demonstrates the most durable activity (FIG. 6). BTEs do not induce off-target toxicity of CA9 non-expressing cells (FIG. 9.)

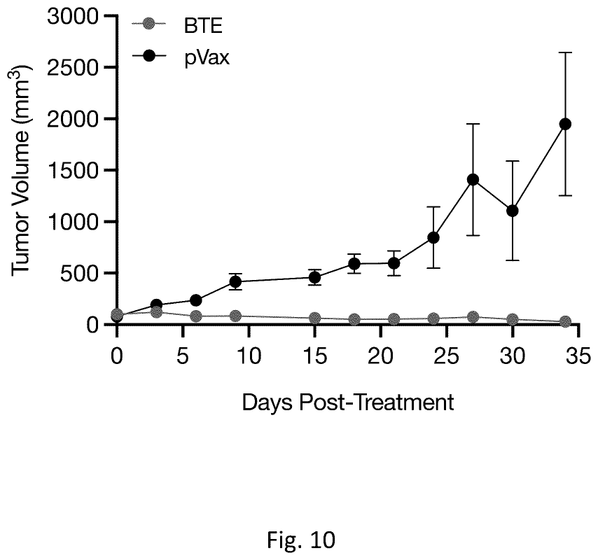

Finally, the data demonstrate that administration of HLV+ BTE results in a reduced tumor volume (FIGS. 5, 6 and 10).

TABLE 1
One-compartment model of BTE pharmacokinetics

| | Compound | | |
|---|---|---|---|
| Parameter | BTE | HLE BTE | HLV+ BTE |
| Cₘₐₓ (ug/mL) | 48.8 ± 26.3 | 22.5 ± 8.9 | 28 ± 11.6 |
| AUC (ug/mL*hr) | 142.1 ± 28.8 | 1786.5 ± 492.9 | 1811.4 ± 1723.4 |
| VD (L) | 1272.9 ± 452.3 | 2685.7 ± 1235.6 | 2152.3 ± 711.3 |
| CL (mL/hr/kg) | 20.6 ± 10.6 | 1.5 ± 0.4 | 2.5 ± 2.4 |
| t_{1/2} (hr) | 3.4 ± 1 | 107.3 ± 19.8 | 92.5 ± 10.7 |

TABLE 2
EC₅₀ (pM) for different treatments

| | | EC₅₀ (pM) | | |
|---|---|---|---|---|
| Cell line | n | BTE | HLE BTE | HLV+ BTE |
| ACHN-CA9 | 3 | 38.7 | 95.3 | 2.3 |
| 293T-CA9 | 2 | 13.2 | 51.3 | 2.3 |
| SKRC-52 | 4 | 1.5 | 14.8 | 0.34 |
| A-498 | 3 | 21.4 | 708.4 | 22.7 |
| 786-O | 2 | 82.5 | 1201 | 55.8 |

TABLE 3
Sequence Information

| SEQ ID NO: | Sequence Type | Description |
|---|---|---|
| SEQ ID NO: 1 | Nucleotide | G37 VL |
| SEQ ID NO: 2 | Amino Acid | G37 VL |
| SEQ ID NO: 3 | Nucleotide | G37 VH |
| SEQ ID NO: 4 | Amino Acid | G37 VH |
| SEQ ID NO: 5 | Nucleotide | CD3 VH |
| SEQ ID NO: 6 | Amino Acid | CD3 VH |
| SEQ ID NO: 7 | Nucleotide | CD3 VL |
| SEQ ID NO: 8 | Amino Acid | CD3 VL |
| SEQ ID NO: 9 | Nucleotide | G37_DBiTE (Non-HLV) |
| SEQ ID NO: 10 | Amino Acid | G37_DBiTE (Non-HLV) |
| SEQ ID NO: 11 | Nucleotide | G37_DBiTE with His tag |
| | | (Non-HLV) |
| SEQ ID NO: 12 | Amino Acid | G37_DBiTE with His tag |
| | | (Non-HLV) |
| SEQ ID NO: 13 | Nucleotide | G37-DBiTE-Fc (HLV-1) |
| SEQ ID NO: 14 | Amino Acid | G37-DBiTE-Fc (HLV-1) |
| SEQ ID NO: 15 | Nucleotide | G37-DBiTE-Fc with His tag (HLV-1) |
| SEQ ID NO: 16 | Amino Acid | G37-DBiTE-Fc with His tag (HLV-1) |
| SEQ ID NO: 17 | Nucleotide | G37-DBiTE-Fc+ (HLV-1) |
| SEQ ID NO: 18 | Amino Acid | G37-DBiTE-Fc+ (HLV-1) |
| SEQ ID NO: 19 | Nucleotide | G37-DBiTE-Fc+ with His tag |
| | | (HLV-1) |
| SEQ ID NO: 20 | Amino Acid | G37-DBiTE-Fc+ with His tag |
| | | (HLV-1) |
| SEQ ID NO: 21 | Nucleotide | G37-TRV-Fc (HLV-2) |
| SEQ ID NO: 22 | Amino Acid | G37-TRV-Fc (HLV-2) |
| SEQ ID NO: 23 | Nucleotide | G37-TRV-Fc with His tag (HLV-2) |
| SEQ ID NO: 24 | Amino Acid | G37-TRV-Fc with His tag (HLV-2) |
| SEQ ID NO: 25 | Nucleotide | G37-TRV-Fc+ (HLV-2) |
| SEQ ID NO: 26 | Amino Acid | G37-TRV-Fc+ (HLV-2) |
| SEQ ID NO: 27 | Nucleotide | G37-TRV-Fc+ with His tag (HLV-2) |
| SEQ ID NO: 28 | Amino Acid | G37-TRV-Fc+ with His tag (HLV-2) |

-G37 VL
SEQ ID NO: 1
Cagagcgtgctgacacagcccccttccgtgtctggagcaccaggacagcgggtgaccatcagctgcac
aggcagctcctctaacatcggcgccggctacgacgtgcactggtatcagcacctgccaggaaccgcaccaaagctgctgatct
acggcaactctaatagacctagcggcgtgccagatcggtttagcggatccaagtctggcaccagcgcctccctggcaatcacag
gactgcaggcagaggacgagacagattacttctgccagtcctatgatagctccctgtctgcctgggtgtttggcggcggaaccaa
ggtgaccgtgctg
-G37 VL
SEQ ID NO: 2
QSVLTQPPSVSGAPGQRVTISCTGSSSNIGAGYDVHWYQHLPGTAP
KLLIYGNSNRPSGVPDRFSGSKSGTSASLAITGLQAEDETDYFCQSYDSSLSAWVF
GGGTKVTVL
-G37 VH
SEQ ID NO: 3
caggtgcagctggtgcagagcggcggcggcgtggtgcagcctggcggctccctgagactgtcttgtgcc
gccagcggcttccctttttctagctacgcaatgagctgggtgcggcaggcaccaggcaagggactggagtgggtgtccgccatc
tctgccaacggcggaaccacatactatgccgactccgtgaagggcaggttcaccatctccagagataactctaagaatacactgt
acctgcagatgaacagcctgagggcagaggacaccgccgtgtactattgcgccaacaatggcaattataggggcgcctttgata
tctggggccagggcaccatggtgacagtgtccagc
-G37 VH
SEQ ID NO: 4
QVQLVQSGGGVVQPGGSLRLSCAASGFPFSSYAMSWVRQAPGKG
LEWVSAISANGGTTYYADSVKGRFTISRDNSKNTLYLQMNSLRAEDTAVYYCAN
NGNYRGAFDIWGQGTMVTVSS
-CD3 VH
SEQ ID NO: 5
Gaggtgcagctggtggagagcggcggcggcctggtgcagccaggcggcagcctgaggctgtcctgtg
cagcaagcggatactccttcaccggctatacaatgaattgggtgaggcaggcccctggcaagggcctggaatgggtggccctg
atcaacccctacaagggcgtgtccacctataatcagaagttcaaggaccgctttaccatctctgtggataagagcaagaacacag
cctacctgcagatgaatagcctgagagccgaggacacagccgtgtactattgcgcacggagcggatactatggcgactccgatt
ggtattttgacgtgtggggccagggcaccctggtgacagtgagcagc
-CD3 VH
SEQ ID NO: 6
EVQLVESGGGLVQPGGSLRLSCAASGYSFTGYTMNWVRQAPGKG
LEWVALINPYKGVSTYNQKFKDRFTISVDKSKNTAYLQMNSLRAEDTAVYYCAR
SGYYGDSDWYFDVWGQGTLVTVSS
-CD3 VL
SEQ ID NO: 7
Gacatccagatgacccagtccccatctagcctgtctgccagcgtcggcgacagggtgaccatcacatgtc
gcgcctctcaggatatcaggaactacctgaattggtatcagcagaagcccggcaaggcccctaagctgctgatctactatacatc
caggctggagtctggagtgccaagcaggttctccggatctggaagcggaaccgactacaccctgacaatctcctctctgcagcc
cgaggatttcgccacatactattgtcagcagggcaataccctgccttggacatttggccagggcaccaaggtggagatcaagag
ctcc
-CD3 VL
SEQ ID NO: 8
DIQMTQSPSSLSASVGDRVTITCRASQDIRNYLNWYQQKPGKAPKL
LIYYTSRLESGVPSRFSGSGSGTDYTLTISSLQPEDFATYYCQQGNTLPWTFGQGT
KVEIKSS
-G37_DBiTE
SEQ ID NO: 9
atggactggacctggatcctgttcctggtggctgccgccaccagagtgcactctcagagcgtgctgacac
agcccccttccgtgtctggagcaccaggacagcgggtgaccatcagctgcacaggcagctcctctaacatcggcgccggctac
gacgtgcactggtatcagcacctgccaggaaccgcaccaaagctgctgatctacggcaactctaatagacctagcggcgtgcc
agatcggtttagcggatccaagtctggcaccagcgcctccctggcaatcacaggactgcaggcagaggacgagacagattact
tctgccagtcctatgatagctccctgtctgcctgggtgtttggcggcggaaccaaggtgaccgtgctgggcggcggcggcagcg
gcggcggcggcagcggcggcggcggctcccaggtgcagctggtgcagagcggcggcggcgtggtgcagcctggcggctc
cctgagactgtcttgtgccgccagcggcttccctttttctagctacgcaatgagctgggtgcggcaggcaccaggcaagggactg
gagtgggtgtccgccatctctgccaacggcggaaccacatactatgccgactccgtgaagggcaggttcaccatctccagagat
aactctaagaatacactgtacctgcagatgaacagcctgagggcagaggacaccgccgtgtactattgcgccaacaatggcaat
tataggggcgcctttgatatctggggccagggcaccatggtgacagtgtccagcggcggcggcggctcggaggtgcagctgg
tggagagcggcggcggcctggtgcagccaggcggcagcctgaggctgtcctgtgcagcaagcggatactccttcaccggcta
tacaatgaattgggtgaggcaggcccctggcaagggcctggaatgggtggccctgatcaacccctacaagggcgtgtccacct
ataatcagaagttcaaggaccgctttaccatctctgtggataagagcaagaacacagcctacctgcagatgaatagcctgagagc
cgaggacacagccgtgtactattgcgcacggagcggatactatggcgactccgattggtattttgacgtgtggggccagggcac
cctggtgacagtgagcagcggcggcggcggcagcggcggcggcggcagcggcggcggcggctctgacatccagatgacc
cagtccccatctagcctgtctgccagcgtcggcgacagggtgaccatcacatgtcgcgcctctcaggatatcaggaactacctga
attggtatcagcagaagcccggcaaggcccctaagctgctgatctactatacatccaggctggagtctggagtgccaagcaggtt
ctccggatctggaagcggaaccgactacaccctgacaatctcctctctgcagcccgaggatttcgccacatactattgtcagcag
ggcaataccctgccttggacatttggccagggcaccaaggtggagatcaagagctcctgataa
-G37_DBiTE
SEQ ID NO: 10
MDWTWILFLVAAATRVHSQSVLTQPPSVSGAPGQRVTISCTGSSSN
IGAGYDVHWYQHLPGTAPKLLIYGNSNRPSGVPDRFSGSKSGTSASLAITGLQAE
DETDYFCQSYDSSLSAWVFGGGTKVTVLGGGGSGGGGSGGGGSQVQLVQSGGG
VVQPGGSLRLSCAASGFPFSSYAMSWVRQAPGKGLEWVSAISANGGTTYYADSV
KGRFTISRDNSKNTLYLQMNSLRAEDTAVYYCANNGNYRGAFDIWGQGTMVTV
SSGGGGSEVQLVESGGGLVQPGGSLRLSCAASGYSFTGYTMNWVRQAPGKGLE
WVALINPYKGVSTYNQKFKDRFTISVDKSKNTAYLQMNSLRAEDTAVYYCARS
GYYGDSDWYFDVWGQGTLVTVSSGGGGSGGGGSGGGGSDIQMTQSPSSLSASV
GDRVTITCRASQDIRNYLNWYQQKPGKAPKLLIYYTSRLESGVPSRFSGSGSGTD
YTLTISSLQPEDFATYYCQQGNTLPWTFGQGTKVEIKSS
-G37_DBiTE with His tag
SEQ ID NO: 11
atggactggacctggatcctgttcctggtggctgccgccaccagagtgcactctcagagcgtgctgacac
agcccccttccgtgtctggagcaccaggacagcgggtgaccatcagctgcacaggcagctcctctaacatcggcgccggctac
gacgtgcactggtatcagcacctgccaggaaccgcaccaaagctgctgatctacggcaactctaatagacctagcggcgtgcc
agatcggtttagcggatccaagtctggcaccagcgcctccctggcaatcacaggactgcaggcagaggacgagacagattact
tctgccagtcctatgatagctccctgtctgcctgggtgtttggcggcggaaccaaggtgaccgtgctgggcggcggcggcagcg
gcggcggcggcagcggcggcggcggctcccaggtgcagctggtgcagagcggcggcggcgtggtgcagcctggcggctc
cctgagactgtcttgtgccgccagcggcttccctttttctagctacgcaatgagctgggtgcggcaggcaccaggcaagggactg
gagtgggtgtccgccatctctgccaacggcggaaccacatactatgccgactccgtgaagggcaggttcaccatctccagagat
aactctaagaatacactgtacctgcagatgaacagcctgagggcagaggacaccgccgtgtactattgcgccaacaatggcaat
tataggggcgcctttgatatctggggccagggcaccatggtgacagtgtccagcggcggcggcggctcggaggtgcagctgg
tggagagcggcggcggcctggtgcagccaggcggcagcctgaggctgtcctgtgcagcaagcggatactccttcaccggcta
tacaatgaattgggtgaggcaggcccctggcaagggcctggaatgggtggccctgatcaacccctacaagggcgtgtccacct
ataatcagaagttcaaggaccgctttaccatctctgtggataagagcaagaacacagcctacctgcagatgaatagcctgagagc
cgaggacacagccgtgtactattgcgcacggagcggatactatggcgactccgattggtattttgacgtgtggggccagggcac
cctggtgacagtgagcagcggcggcggcggcagcggcggcggcggcagcggcggcggcggctctgacatccagatgacc
cagtccccatctagcctgtctgccagcgtcggcgacagggtgaccatcacatgtcgcgcctctcaggatatcaggaactacctga
attggtatcagcagaagcccggcaaggcccctaagctgctgatctactatacatccaggctggagtctggagtgccaagcaggtt
ctccggatctggaagcggaaccgactacaccctgacaatctcctctctgcagcccgaggatttcgccacatactattgtcagcag
ggcaataccctgccttggacatttggccagggcaccaaggtggagatcaagagctcccaccaccaccaccaccactgataa
-G37_DBiTE with His tag
SEQ ID NO: 12
MDWTWILFLVAAATRVHSQSVLTQPPSVSGAPGQRVTISCTGSSSNIGAGYDVH
WYQHLPGTAPKLLIYGNSNRPSGVPDRFSGSKSGTSASLAITGLQAEDETDYFCQ
SYDSSLSAWVFGGGTKVTVLGGGGSGGGGSGGGGSQVQLVQSGGGVVQPGGSL
RLSCAASGFPFSSYAMSWVRQAPGKGLEWVSAISANGGTTYYADSVKGRFTISR
DNSKNTLYLQMNSLRAEDTAVYYCANNGNYRGAFDIWGQGTMVTVSSGGGGS
EVQLVESGGGLVQPGGSLRLSCAASGYSFTGYTMNWVRQAPGKGLEWVALINP
YKGVSTYNQKFKDRFTISVDKSKNTAYLQMNSLRAEDTAVYYCARSGYYGDSD
WYFDVWGQGTLVTVSSGGGGSGGGGSGGGGSDIQMTQSPSSLSASVGDRVTITC
RASQDIRNYLNWYQQKPGKAPKLLIYYTSRLESGVPSRFSGSGSGTDYTLTISSLQ
PEDFATYYCQQGNTLPWTFGQGTKVEIKSSHHHHHH
-G37-DBITE-Fc
SEQ ID NO: 13
ATGGACTGGACCTGGATCCTGTTCCTGGTGGCTGCCGCCACCAG
GGTGCACAGCGACAAGACCCACACATGCCCTCCATGTCCAGCACCAGAGGCA
GCCGGCGGACCTAGCGTGTTCCTGTTTCCCCCTAAGCCAAAGGATACCCTGAT
GATCTCTCGGACCCCTGAGGTGACATGCGTGGTGGTGGACGTGAGCCACGAG
GCACCAGAGGTGAAGTTCAACTGGTACGTGGATGGCGTGGAGGTGCACAATG
CCAAGACAAAGCCTTGCGAGGAGCAGTACGGCTCCACCTATCGCTGCGTGAG
CGTGCTGACAGTGCTGCACCAGGACTGGCTGAACGGCAAGGAGTATAAGTGC
GCCGTGTCCAATAAGGCCCTGCCTGCCCCAATCGAGAAGACCATCTCTAAGG
CCAAGGGCCAGCCTAGGGAGCCACAGGTGTACACACTGCCACCCTCCAGAGA
GGAGATGACCAAGAACCAGGTGTCTCTGACATGTCTGGTGAAGGGCTTCTAT
CCCAGCGACATCGCCGTGGAGTGGGAGTCCAATGGCCAGCCTGAGAACAATT
ACAAGACCACACCTCCAGTGCTGGACTCTGATGGCAGCTTCTTTCTGTATTCT
AAGCTGACCGTGGATAAGAGCCGGTGGCAGCAGGGCAACGTGTTTAGCTGCT
CCGTGATGCACGAGGCCCTGCACAATCACTACACACAGAAGTCTCTGAGCCT
GTCCCCAGGCAAGGGCGGCGGCGGCAGCGGCGGCGGCGGCAGCGGCGGCGG
CGGCAGCGGCGGCGGCGGCAGCGGCGGCGGCGGCAGCGGCGGCGGCGGCTC
GGATAAAACCCACACATGCCCTCCCTGTCCAGCACCTGAGGCAGCCGGCGGA
CCAAGCGTGTTCCTGTTTCCACCCAAGCCTAAAGACACCCTGATGATCAGCAG
GACCCCCGAAGTCACCTGCGTGGTCGTGGACGTGTCCCACGAGGCACCTGAA
GTCAAGTTCAACTGGTACGTGGACGGAGTCGAAGTCCATAACGCCAAGACAA
AGCCCTGTGAAGAGCAGTACGGCAGCACCTATAGATGCGTGAGCGTGCTGAC
AGTGCTGCATCAGGATTGGCTGAATGGCAAGGAATACAAGTGCGCCGTGTCT
AATAAGGCCCTGCCAGCCCCCATCGAGAAGACCATCTCCAAGGCAAAGGGAC
AGCCAAGGGAGCCTCAGGTGTACACACTGCCTCCAAGCCGCGAAGAAATGAC
TAAAAACCAGGTGTCCCTGACCTGCCTGGTCAAAGGCTTCTATCCATCTGATA
TTGCCGTGGAGTGGGAGAGCAATGGCCAGCCCGAAAATAATTACAAGACCAC
ACCCCCTGTGCTGGACTCCGATGGCTCTTTCTTTCTGTATTCCAAACTGACTGT
GGATAAGTCTCGCTGGCAGCAGGGAAACGTGTTTTCTTGTAGCGTGATGCATG
AGGCCCTGCATAACCACTACACACAGAAGTCCCTGTCTCTGAGCCCTGGCAA
GGGCGGCGGCGGCAGCGGCGGCGGCGGCtctcagagcgtgctgacacagcccccttccgtgtctgga
gcaccaggacagcgggtgaccatcagctgcacaggcagctcctctaacatcggcgccggctacgacgtgcactggtatcagc
acctgccaggaaccgcaccaaagctgctgatctacggcaactctaatagacctagcggcgtgccagatcggtttagcggatcca
agtctggcaccagcgcctccctggcaatcacaggactgcaggcagaggacgagacagattacttctgccagtcctatgatagct
ccctgtctgcctgggtgtttggcggcggaaccaaggtgaccgtgctgggcggcggcggcagcggcggcggcggcagcggc
ggcggcggctcccaggtgcagctggtgcagagcggcggcggcgtggtgcagcctggcggctccctgagactgtcttgtgccg
ccagcggcttccctttttctagctacgcaatgagctgggtgcggcaggcaccaggcaagggactggagtgggtgtccgccatct
ctgccaacggcggaaccacatactatgccgactccgtgaagggcaggttcaccatctccagagataactctaagaatacactgta
cctgcagatgaacagcctgagggcagaggacaccgccgtgtactattgcgccaacaatggcaattataggggcgcctttgatat
ctggggccagggcaccatggtgacagtgtccagcggcggcggcggctcggaggtgcagctggtggagagcggcggcggc
ctggtgcagccaggcggcagcctgaggctgtcctgtgcagcaagcggatactccttcaccggctatacaatgaattgggtgagg
caggcccctggcaagggcctggaatgggtggccctgatcaacccctacaagggcgtgtccacctataatcagaagttcaagga
ccgctttaccatctctgtggataagagcaagaacacagcctacctgcagatgaatagcctgagagccgaggacacagccgtgta
ctattgcgcacggagcggatactatggcgactccgattggtattttgacgtgtggggccagggcaccctggtgacagtgagcag
cggcggcggcggcagcggcggcggcggcagcggcggcggcggctctgacatccagatgacccagtccccatctagcctgt
ctgccagcgtcggcgacagggtgaccatcacatgtcgcgcctctcaggatatcaggaactacctgaattggtatcagcagaagc
ccggcaaggcccctaagctgctgatctactatacatccaggctggagtctggagtgccaagcaggttctccggatctggaagcg
gaaccgactacaccctgacaatctcctctctgcagcccgaggatttcgccacatactattgtcagcagggcaataccctgccttgg
acatttggccagggcaccaaggtggagatcaagagctcctgataa
-G37-DBITE-Fc
SEQ ID NO: 14
MDWTWILFLVAAATRVHSDKTHTCPPCPAPEAAGGPSVFLFPPKP
KDTLMISRTPEVTCVVVDVSHEAPEVKFNWYVDGVEVHNAKTKPCEEQYGSTY
RCVSVLTVLHQDWLNGKEYKCAVSNKALPAPIEKTISKAKGQPREPQVYTLPPSR
EEMTKNQVSLTCLVKGFYPSDIAVEWESNGQPENNYKTTPPVLDSDGSFFLYSKL
TVDKSRWQQGNVFSCSVMHEALHNHYTQKSLSLSPGKGGGGSGGGGSGGGGSG
GGGSGGGGSGGGGSDKTHTCPPCPAPEAAGGPSVFLFPPKPKDTLMISRTPEVTC
VVVDVSHEAPEVKFNWYVDGVEVHNAKTKPCEEQYGSTYRCVSVLTVLHQDW
LNGKEYKCAVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNV
FSCSVMHEALHNHYTQKSLSLSPGKGGGGSGGGGSQSVLTQPPSVSGAPGQRVTI
SCTGSSSNIGAGYDVHWYQHLPGTAPKLLIYGNSNRPSGVPDRFSGSKSGTSASL
AITGLQAEDETDYFCQSYDSSLSAWVFGGGTKVTVLGGGGSGGGGSGGGGSQV
QLVQSGGGVVQPGGSLRLSCAASGFPFSSYAMSWVRQAPGKGLEWVSAISANG
GTTYYADSVKGRFTISRDNSKNTLYLQMNSLRAEDTAVYYCANNGNYRGAFDI
WGQGTMVTVSSGGGGSEVQLVESGGGLVQPGGSLRLSCAASGYSFTGYTMNW
VRQAPGKGLEWVALINPYKGVSTYNQKFKDRFTISVDKSKNTAYLQMNSLRAED
TAVYYCARSGYYGDSDWYFDVWGQGTLVTVSSGGGGSGGGGSGGGGSDIQMT
QSPSSLSASVGDRVTITCRASQDIRNYLNWYQQKPGKAPKLLIYYTSRLESGVPSR
FSGSGSGTDYTLTISSLQPEDFATYYCQQGNTLPWTFGQGTKVEIKSS
-G37-DBiTE-Fc with His tag
SEQ ID NO: 15
ATGGACTGGACCTGGATCCTGTTCCTGGTGGCTGCCGCCACCAG
GGTGCACAGCGACAAGACCCACACATGCCCTCCATGTCCAGCACCAGAGGCA
GCCGGCGGACCTAGCGTGTTCCTGTTTCCCCCTAAGCCAAAGGATACCCTGAT
GATCTCTCGGACCCCTGAGGTGACATGCGTGGTGGTGGACGTGAGCCACGAG
GCACCAGAGGTGAAGTTCAACTGGTACGTGGATGGCGTGGAGGTGCACAATG
CCAAGACAAAGCCTTGCGAGGAGCAGTACGGCTCCACCTATCGCTGCGTGAG
CGTGCTGACAGTGCTGCACCAGGACTGGCTGAACGGCAAGGAGTATAAGTGC
GCCGTGTCCAATAAGGCCCTGCCTGCCCCAATCGAGAAGACCATCTCTAAGG
CCAAGGGCCAGCCTAGGGAGCCACAGGTGTACACACTGCCACCCTCCAGAGA
GGAGATGACCAAGAACCAGGTGTCTCTGACATGTCTGGTGAAGGGCTTCTAT
CCCAGCGACATCGCCGTGGAGTGGGAGTCCAATGGCCAGCCTGAGAACAATT
ACAAGACCACACCTCCAGTGCTGGACTCTGATGGCAGCTTCTTTCTGTATTCT
AAGCTGACCGTGGATAAGAGCCGGTGGCAGCAGGGCAACGTGTTTAGCTGCT
CCGTGATGCACGAGGCCCTGCACAATCACTACACACAGAAGTCTCTGAGCCT
GTCCCCAGGCAAGGGCGGCGGCGGCAGCGGCGGCGGCGGCAGCGGCGGCGG
CGGCAGCGGCGGCGGCGGCAGCGGCGGCGGCGGCAGCGGCGGCGGCGGCTC
GGATAAAACCCACACATGCCCTCCCTGTCCAGCACCTGAGGCAGCCGGCGGA
CCAAGCGTGTTCCTGTTTCCACCCAAGCCTAAAGACACCCTGATGATCAGCAG
GACCCCCGAAGTCACCTGCGTGGTCGTGGACGTGTCCCACGAGGCACCTGAA
GTCAAGTTCAACTGGTACGTGGACGGAGTCGAAGTCCATAACGCCAAGACAA
AGCCCTGTGAAGAGCAGTACGGCAGCACCTATAGATGCGTGAGCGTGCTGAC
AGTGCTGCATCAGGATTGGCTGAATGGCAAGGAATACAAGTGCGCCGTGTCT
AATAAGGCCCTGCCAGCCCCCATCGAGAAGACCATCTCCAAGGCAAAGGGAC
AGCCAAGGGAGCCTCAGGTGTACACACTGCCTCCAAGCCGCGAAGAAATGAC
TAAAAACCAGGTGTCCCTGACCTGCCTGGTCAAAGGCTTCTATCCATCTGATA
TTGCCGTGGAGTGGGAGAGCAATGGCCAGCCCGAAAATAATTACAAGACCAC
ACCCCCTGTGCTGGACTCCGATGGCTCTTTCTTTCTGTATTCCAAACTGACTGT
GGATAAGTCTCGCTGGCAGCAGGGAAACGTGTTTTCTTGTAGCGTGATGCATG
AGGCCCTGCATAACCACTACACACAGAAGTCCCTGTCTCTGAGCCCTGGCAA
GGGCGGCGGCGGCAGCGGCGGCGGCGGCtctcagagcgtgctgacacagcccccttccgtgtctgga
gcaccaggacagcgggtgaccatcagctgcacaggcagctcctctaacatcggcgccggctacgacgtgcactggtatcagc
acctgccaggaaccgcaccaaagctgctgatctacggcaactctaatagacctagcggcgtgccagatcggtttagcggatcca
agtctggcaccagcgcctccctggcaatcacaggactgcaggcagaggacgagacagattacttctgccagtcctatgatagct
ccctgtctgcctgggtgtttggcggcggaaccaaggtgaccgtgctgggcggcggcggcagcggcggcggcggcagcggc
ggcggcggctcccaggtgcagctggtgcagagcggcggcggcgtggtgcagcctggcggctccctgagactgtcttgtgccg
ccagcggcttccctttttctagctacgcaatgagctgggtgcggcaggcaccaggcaagggactggagtgggtgtccgccatct
ctgccaacggcggaaccacatactatgccgactccgtgaagggcaggttcaccatctccagagataactctaagaatacactgta
cctgcagatgaacagcctgagggcagaggacaccgccgtgtactattgcgccaacaatggcaattataggggcgcctttgatat
ctggggccagggcaccatggtgacagtgtccagcggcggcggcggctcggaggtgcagctggtggagagcggcggcggc
ctggtgcagccaggcggcagcctgaggctgtcctgtgcagcaagcggatactccttcaccggctatacaatgaattgggtgagg
caggcccctggcaagggcctggaatgggtggccctgatcaacccctacaagggcgtgtccacctataatcagaagttcaagga
ccgctttaccatctctgtggataagagcaagaacacagcctacctgcagatgaatagcctgagagccgaggacacagccgtgta
ctattgcgcacggagcggatactatggcgactccgattggtattttgacgtgtggggccagggcaccctggtgacagtgagcag
cggcggcggcggcagcggcggcggcggcagcggcggcggcggctctgacatccagatgacccagtccccatctagcctgt
ctgccagcgtcggcgacagggtgaccatcacatgtcgcgcctctcaggatatcaggaactacctgaattggtatcagcagaagc
ccggcaaggcccctaagctgctgatctactatacatccaggctggagtctggagtgccaagcaggttctccggatctggaagcg
gaaccgactacaccctgacaatctcctctctgcagcccgaggatttcgccacatactattgtcagcagggcaataccctgccttgg
acatttggccagggcaccaaggtggagatcaagagctcccaccaccaccaccaccactgataa
-G37-DBiTE-Fc with His tag
SEQ ID NO: 16
MDWTWILFLVAAATRVHSDKTHTCPPCPAPEAAGGPSVFLFPPKP
KDTLMISRTPEVTCVVVDVSHEAPEVKFNWYVDGVEVHNAKTKPCEEQYGSTY
RCVSVLTVLHQDWLNGKEYKCAVSNKALPAPIEKTISKAKGQPREPQVYTLPPSR
EEMTKNQVSLTCLVKGFYPSDIAVEWESNGQPENNYKTTPPVLDSDGSFFLYSKL
TVDKSRWQQGNVFSCSVMHEALHNHYTQKSLSLSPGKGGGGSGGGGSGGGGSG
GGGSGGGGSGGGGSDKTHTCPPCPAPEAAGGPSVFLFPPKPKDTLMISRTPEVTC
VVVDVSHEAPEVKFNWYVDGVEVHNAKTKPCEEQYGSTYRCVSVLTVLHQDW
LNGKEYKCAVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNV
FSCSVMHEALHNHYTQKSLSLSPGKGGGGSGGGGSQSVLTQPPSVSGAPGQRVTI
SCTGSSSNIGAGYDVHWYQHLPGTAPKLLIYGNSNRPSGVPDRFSGSKSGTSASL
AITGLQAEDETDYFCQSYDSSLSAWVFGGGTKVTVLGGGGGGGGSGGGGSQV
QLVQSGGGVVQPGGSLRLSCAASGFPFSSYAMSWVRQAPGKGLEWVSAISANG
GTTYYADSVKGRFTISRDNSKNTLYLQMNSLRAEDTAVYYCANNGNYRGAFDI
WGQGTMVTVSSGGGGSEVQLVESGGGLVQPGGSLRLSCAASGYSFTGYTMNW
VRQAPGKGLEWVALINPYKGVSTYNQKFKDRFTISVDKSKNTAYLQMNSLRAED
TAVYYCARSGYYGDSDWYFDVWGQGTLVTVSSGGGGSGGGGSGGGGSDIQMT
QSPSSLSASVGDRVTITCRASQDIRNYLNWYQQKPGKAPKLLIYYTSRLESGVPSR
FSGSGSGTDYTLTISSLQPEDFATYYCQQGNTLPWTFGQGTKVEIKSSHHHHHH
-G37-DBiTE-Fc+ (with mutations for half life extension)
SEQ ID NO: 17
atggactggacctggatcctgttcctggtggctgccgccaccagggtgcacagcgacaagacccacacatgccctccatgtccagcaccag
aggcagccggcggacctagcgtgttcctgtttccccctaagccaaaggataccctgatgatctctcggacccctgaggtgacatgcgtggtg
gtggacgtgagccacgaggcaccagaggtgaagttcaactggtacgtggatggcgtggaggtgcacaatgccaagacaaagccttgcga
ggagcagtacggctccacctatcgctgcgtgagcgtgctgacagtgctgcaccaggactggctgaacggcaaggagtataagtgcgccgt
gtccaataaggccctgcctgccccaatcgagaagaccatctctaaggccaagggccagcctagggagccacaggtgtacacactgccacc
ctccagagaggagatgaccaagaaccaggtgtctctgacatgtctggtgaagggcttctatcccagcgacatcgccgtggagtgggagtcc
aatggccagcctgagaacaattacaagaccacacctccagtgctggactctgatggcagcttctttctgtattctaagctgaccgtggataag
agccggtggcagcagggcaacgtgtttagctgctccgtgctgcacgaggccctgcactctcactacacacagaagtctctgagcctgtcccca
ggcaagggcggcggcggcagcggcggcggcggcagcggcggcggcggcagcggcggcggcggcagcggcggcggcggcagcg
gcggcggcggctcggataaaacccacacatgccctccctgtccagcacctgaggcagccggcggaccaagcgtgttcctgtttccaccca
agcctaaagacaccctgatgatcagcaggacccccgaagtcacctgcgtggtcgtggacgtgtcccacgaggcacctgaagtcaagttcaa
ctggtacgtggacggagtcgaagtccataacgccaagacaaagccctgtgaagagcagtacggcagcacctatagatgcgtgagcgtgct
gacagtgctgcatcaggattggctgaatggcaaggaatacaagtgcgccgtgtctaataaggccctgccagcccccatcgagaagaccatc
tccaaggcaaagggacagccaagggagcctcaggtgtacacactgcctccaagccgcgaagaaatgactaaaaaccaggtgtccctgac
ctgcctggtcaaaggcttctatccatctgatattgccgtggagtgggagagcaatggccagcccgaaaataattacaagaccacaccccctgt
gctggactccgatggctctttctttctgtattccaaactgactgtggataagtctcgctggcagcagggaaacgtgttttcttgtagcgtgct
gcacgaggccctgcactctcactacacacagaagtccctgtctctgagccctggcaagggcggcggcggcagcggcggcggcggctctcaga
gcgtgctgacacagcccccttccgtgtctggagcaccaggacagcgggtgaccatcagctgcacaggcagctcctctaacatc
ggcgccggctacgacgtgcactggtatcagcacctgccaggaaccgcaccaaagctgctgatctacggcaactctaatagac
ctagcggcgtgccagatcggtttagcggatccaagtctggcaccagcgcctccctggcaatcacaggactgcaggcagagga
cgagacagattacttctgccagtcctatgatagctccctgtctgcctgggtgtttggcggcggaaccaaggtgaccgtgctggg
cggcggcggcagcggcggcggcggcagcggcggcggcggctcccaggtgcagctggtgcagagcggcggcggcgtggtgc
agcctggcggctccctgagactgtcttgtgccgccagcggcttccctttttctagctacgcaatgagctgggtgcggcaggcacc
aggcaagggactggagtgggtgtccgccatctctgccaacggcggaaccacatactatgccgactccgtgaagggcaggttc
accatctccagagataactctaagaatacactgtacctgcagatgaacagcctgagggcagaggacaccgccgtgtactattg
cgccaacaatggcaattataggggcgcctttgatatctggggccagggcaccatggtgacagtgtccagcggcggcggcggct
cggaggtgcagctggtggagagcggcggcggcctggtgcagccaggcggcagcctgaggctgtcctgtgcagcaagcggat
actccttcaccggctatacaatgaattgggtgaggcaggcccctggcaagggcctggaatgggtggccctgatcaacccctac
aagggcgtgtccacctataatcagaagttcaaggaccgctttaccatctctgtggataagagcaagaacacagcctacctgca
gatgaatagcctgagagccgaggacacagccgtgtactattgcgcacggagcggatactatggcgactccgattggtattttg
acgtgtggggccagggcaccctggtgacagtgagcagcggcggcggcggcagcggcggcggcggcagcggcggcggcggc
tctgacatccagatgacccagtccccatctagcctgtctgccagcgtcggcgacagggtgaccatcacatgtcgcgcctctcag
gatatcaggaactacctgaattggtatcagcagaagcccggcaaggcccctaagctgctgatctactatacatccaggctgga
gtctggagtgccaagcaggttctccggatctggaagcggaaccgactacaccctgacaatctcctctctgcagcccgaggattt
cgccacatactattgtcagcagggcaataccctgccttggacatttggccagggcaccaaggtggagatcaagagctcctgat
aa
-G37-DBiTE-Fc+ (with mutations for half life extension)
SEQ ID NO: 18
MDWTWILFLVAAATRVHSDKTHTCPPCPAPEAAGGPSVFLFPPKP
KDTLMISRTPEVTCVVVDVSHEAPEVKFNWYVDGVEVHNAKTKPCEEQYGSTY
RCVSVLTVLHQDWLNGKEYKCAVSNKALPAPIEKTISKAKGQPREPQVYTLPPSR
EEMTKNQVSLTCLVKGFYPSDIAVEWESNGQPENNYKTTPPVLDSDGSFFLYSKL
TVDKSRWQQGNVFSCSVLHEALHSHYTQKSLSLSPGKGGGGSGGGGSGGGGSG
GGGSGGGGSGGGGSDKTHTCPPCPAPEAAGGPSVFLFPPKPKDTLMISRTPEVTC
VVVDVSHEAPEVKFNWYVDGVEVHNAKTKPCEEQYGSTYRCVSVLTVLHQDW
LNGKEYKCAVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNV
FSCSVLHEALHSHYTQKSLSLSPGKGGGGSGGGGSQSVLTQPPSVSGAPGQRVTI
SCTGSSSNIGAGYDVHWYQHLPGTAPKLLIYGNSNRPSGVPDRFSGSKSGTSASL
AITGLQAEDETDYFCQSYDSSLSAWVFGGGTKVTVLGGGGSGGGGSGGGGSQV
QLVQSGGGVVQPGGSLRLSCAASGFPFSSYAMSWVRQAPGKGLEWVSAISANG
GTTYYADSVKGRFTISRDNSKNTLYLQMNSLRAEDTAVYYCANNGNYRGAFDI
WGQGTMVTVSSGGGGSEVQLVESGGGLVQPGGSLRLSCAASGYSFTGYTMNW
VRQAPGKGLEWVALINPYKGVSTYNQKFKDRFTISVDKSKNTAYLQMNSLRAED
TAVYYCARSGYYGDSDWYFDVWGQGTLVTVSSGGGGSGGGGSGGGGSDIQMT
QSPSSLSASVGDRVTITCRASQDIRNYLNWYQQKPGKAPKLLIYYTSRLESGVPSR
FSGSGSGTDYTLTISSLQPEDFATYYCQQGNTLPWTFGQGTKVEIKSS
-G37-DBiTE-Fc+ with His tag (with mutations for half life extension)
SEQ ID NO: 19
atggactggacctggatcctgttcctggtggctgccgccaccagggtgcacagcgacaagacccacaca
tgccctccatgtccagcaccagaggcagccggcggacctagcgtgttcctgtttccccctaagccaaaggataccctgatgatct
ctcggacccctgaggtgacatgcgtggtggtggacgtgagccacgaggcaccagaggtgaagttcaactggtacgtggatgg
cgtggaggtgcacaatgccaagacaaagccttgcgaggagcagtacggctccacctatcgctgcgtgagcgtgctgacagtgc
tgcaccaggactggctgaacggcaaggagtataagtgcgccgtgtccaataaggccctgcctgccccaatcgagaagaccatc
tctaaggccaagggccagcctagggagccacaggtgtacacactgccaccctccagagaggagatgaccaagaaccaggtgt
ctctgacatgtctggtgaagggcttctatcccagcgacatcgccgtggagtgggagtccaatggccagcctgagaacaattacaa
gaccacacctccagtgctggactctgatggcagcttctttctgtattctaagctgaccgtggataagagccggtggcagcagggc
aacgtgtttagctgctccgtgctgcacgaggccctgcactctcactacacacagaagtctctgagcctgtccccaggcaagggc
ggcggcggcagcggcggcggcggcagcggcggcggcggcagcggcggcggcggcagcggcggcggcggcagcggc
ggcggcggctcggataaaacccacacatgccctccctgtccagcacctgaggcagccggcggaccaagcgtgttcctgtttcc
acccaagcctaaagacaccctgatgatcagcaggacccccgaagtcacctgcgtggtcgtggacgtgtcccacgaggcacctg
aagtcaagttcaactggtacgtggacggagtcgaagtccataacgccaagacaaagccctgtgaagagcagtacggcagcac
ctatagatgcgtgagcgtgctgacagtgctgcatcaggattggctgaatggcaaggaatacaagtgcgccgtgtctaataaggcc
ctgccagcccccatcgagaagaccatctccaaggcaaagggacagccaagggagcctcaggtgtacacactgcctccaagcc
gcgaagaaatgactaaaaaccaggtgtccctgacctgcctggtcaaaggcttctatccatctgatattgccgtggagtgggagag
caatggccagcccgaaaataattacaagaccacaccccctgtgctggactccgatggctcttttttctgtattccaaactgactgt
ggataagtctcgctggcagcagggaaacgtgttttcttgtagcgtgctgcacgaggccctgcactctcactacacacagaagtcc
ctgtctctgagccctggcaagggcggcggcggcagcggcggcggcggctctcagagcgtgctgacacagcccccttccgtgt
ctggagcaccaggacagcgggtgaccatcagctgcacaggcagctcctctaacatcggcgccggctacgacgtgcactggta
tcagcacctgccaggaaccgcaccaaagctgctgatctacggcaactctaatagacctagcggcgtgccagatcggtttagcg
gatccaagtctggcaccagcgcctccctggcaatcacaggactgcaggcagaggacgagacagattacttctgccagtcctat
gatagctccctgtctgcctgggtgtttggcggcggaaccaaggtgaccgtgctgggcggcggcggcagcggcggcggcggca
gcggcggcggcggctcccaggtgcagctggtgcagagcggcggcggcgtggtgcagcctggcggctccctgagactgtcttgt
gccgccagcggcttccctttttctagctacgcaatgagctgggtgcggcaggcaccaggcaagggactggagtgggtgtccgc
catctctgccaacggcggaaccacatactatgccgactccgtgaagggcaggttcaccatctccagagataactctaagaata
cactgtacctgcagatgaacagcctgagggcagaggacaccgccgtgtactattgcgccaacaatggcaattataggggcgc
ctttgatatctggggccagggcaccatggtgacagtgtccagcggcggcggcggctcggaggtgcagctggtggagagcggc
ggcggcctggtgcagccaggcggcagcctgaggctgtcctgtgcagcaagcggatactccttcaccggctatacaatgaattg
ggtgaggcaggcccctggcaagggcctggaatgggtggccctgatcaacccctacaagggcgtgtccacctataatcagaag
ttcaaggaccgctttaccatctctgtggataagagcaagaacacagcctacctgcagatgaatagcctgagagccgaggaca
cagccgtgtactattgcgcacggagcggatactatggcgactccgattggtattttgacgtgtggggccagggcaccctggtga
cagtgagcagcggcggcggcggcagcggcggcggcggcagcggcggcggcggctctgacatccagatgacccagtccccat
ctagcctgtctgccagcgtcggcgacagggtgaccatcacatgtcgcgcctctcaggatatcaggaactacctgaattggtatc
agcagaagcccggcaaggcccctaagctgctgatctactatacatccaggctggagtctggagtgccaagcaggttctccgga
tctggaagcggaaccgactacaccctgacaatctcctctctgcagcccgaggatttcgccacatactattgtcagcagggcaat
accctgccttggacatttggccagggcaccaaggtggagatcaagagctcccaccaccaccaccaccactgataa
-G37-DBITE-Fc+ with His tag (with mutations for half life extension)
SEQ ID NO: 20
MDWTWILFLVAAATRVHSDKTHTCPPCPAPEAAGGPSVFLFPPKP
KDTLMISRTPEVTCVVVDVSHEAPEVKFNWYVDGVEVHNAKTKPCEEQYGSTY
RCVSVLTVLHQDWLNGKEYKCAVSNKALPAPIEKTISKAKGQPREPQVYTLPPSR
EEMTKNQVSLTCLVKGFYPSDIAVEWESNGQPENNYKTTPPVLDSDGSFFLYSKL
TVDKSRWQQGNVFSCSVLHEALHSHYTQKSLSLSPGKGGGGSGGGGSGGGGSG
GGGSGGGGSGGGGSDKTHTCPPCPAPEAAGGPSVFLFPPKPKDTLMISRTPEVTC
VVVDVSHEAPEVKFNWYVDGVEVHNAKTKPCEEQYGSTYRCVSVLTVLHQDW
LNGKEYKCAVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNV
FSCSVLHEALHSHYTQKSLSLSPGKGGGGSGGGGSQSVLTQPPSVSGAPGQRVTI
SCTGSSSNIGAGYDVHWYQHLPGTAPKLLIYGNSNRPSGVPDRFSGSKSGTSASL
AITGLQAEDETDYFCQSYDSSLSAWVFGGGTKVTVLGGGGSGGGGGGGGSQV
QLVQSGGGVVQPGGSLRLSCAASGFPFSSYAMSWVRQAPGKGLEWVSAISANG
GTTYYADSVKGRFTISRDNSKNTLYLQMNSLRAEDTAVYYCANNGNYRGAFDI
WGQGTMVTVSSGGGGSEVQLVESGGGLVQPGGSLRLSCAASGYSFTGYTMNW
VRQAPGKGLEWVALINPYKGVSTYNQKFKDRFTISVDKSKNTAYLQMNSLRAED
TAVYYCARSGYYGDSDWYFDVWGQGTLVTVSSGGGGSGGGGSGGGGSDIQMT
QSPSSLSASVGDRVTITCRASQDIRNYLNWYQQKPGKAPKLLIYYTSRLESGVPSR
FSGSGSGTDYTLTISSLQPEDFATYYCQQGNTLPWTFGQGTKVEIKSSHHHHHH
-G37-TRV-Fc
SEQ ID NO: 21
atggactggacctggatcctgttcctggtggctgccgccacaagggtgcacagcgataagacccacacat
gccctccctgtcctgcaccagaggcagccggcggaccttccgtgttcctgtttccacccaagccaaaggacaccctgatgatca
gcagaacccccgaggtgacatgcgtggtggtggacgtgtcccacgaggcacctgaggtgaagtttaactggtacgtggatggc
gtggaggtgcacaatgccaagacaaagccctgcgaggagcagtacggctctacctatagatgcgtgagcgtgctgacagtgct
gcaccaggattggctgaacggcaaggagtataagtgcgccgtgtctaataaggccctgcccgcccctatcgagaagaccatca
gcaaggcaaagggacagccaagggagcctcaggtgtacacactgcctccatctagagaggagatgaccaagaaccaggtga
gcctgacatgtctggtgaagggcttctatccatccgacatcgccgtggagtgggagtctaatggccagcccgagaacaattacaa
gaccacaccccctgtgctggactccgatggctctttctttctgtatagcaagctgaccgtggataagtccaggtggcagcagggc
aacgtgtttagctgctccgtgatgcacgaggccctgcacaatcactacacacagaagtctctgagcctgtcccctggcaagggc
ggcggcggcagcggcggcggcggcagcggcggcggcggcagcggcggcggcggcagcggcggcggcggcagcggc
ggcggcggctccgacaagacccacacatgcccaccctgtccagcccctgaggcagccggcggaccaagcgtgttcctgtttc
ctccaaagcctaaggataccctgatgatctcccggaccccagaagtcacctgcgtggtcgtggacgtgtctcacgaggcccccg
aggtgaagttcaactggtacgtggacggagtcgaagtgcacaatgccaagaccaagccatgtgaagagcagtacggctccacc
tatcgctgcgtgagcgtgctgacagtgctgcatcaggactggctgaatggcaaggaatataagtgcgccgtgagcaataaggcc
ctgcctgccccaatcgagaagaccatctctaaagccaaaggacagccaagggagccacaggtgtacacactgcccccttcccg
cgaagaaatgactaaaaaccaggtgtctctgacctgcctggtcaaaggcttctatcctagcgacatcgcagtggagtgggagtcc
aacggacagccagaaaataattacaagaccacaccacccgtgctggacagcgatggctccttctttctgtattctaaactgactgt
ggataagagcagatggcagcagggaaacgtgttttcttgtagcgtgatgcatgaggccctgcataaccactacacacagaagtc
cctgtctctgagtccaggcaagggcggcggcggcagcggcggcggcggctctcagagcgtgctgacacagcccccttccgt
gtctggagcaccaggacagcgggtgaccatcagctgcacaggcagctcctctaacatcggcgccggctacgacgtgcactgg
tatcagcacctgccaggaaccgcaccaaagctgctgatctacggcaactctaatagacctagcggcgtgccagatcggtttag
cggatccaagtctggcaccagcgcctccctggcaatcacaggactgcaggcagaggacgagacagattacttctgccagtcct
atgatagctccctgtctgcctgggtgtttggcggcggaaccaaggtgaccgtgctgggcggcggcggcagcggcggcggcgg
cagcggcggcggcggctcccaggtgcagctggtgcagagcggcggcggcgtggtgcagcctggcggctccctgagactgtct
tgtgccgccagcggcttccctttttctagctacgcaatgagctgggtgcggcaggcaccaggcaagggactggagtgggtgtcc
gccatctctgccaacggcggaaccacatactatgccgactccgtgaagggcaggttcaccatctccagagataactctaagaa
tacactgtacctgcagatgaacagcctgagggcagaggacaccgccgtgtactattgcgccaacaatggcaattataggggc
gcctttgatatctggggccagggcaccatggtgacagtgtccagcggcggcggcggctcggaggtgcagctggtggagagcg
gcggcggcctggtgcagccaggcggcagcctgaggctgtcctgtgcagcaagcggatactccttcaccggctatacaatgaat
tgggtgaggcaggcccctggcaagggcctggaatgggtggccctgatcaacccctacaagggcgtgtccacctataatcaga
agttcaaggaccgctttaccatctctgtggataagagcaagaacacagcctacctgcagatgaatagcctgagagccgagga
cacagccgtgtactattgcgcacggagcggatactatggcgactccgattggtattttgacgtgtggggccagggcaccctggt
gacagtgagcagcggcggcggcggcagcggcggcggcggcagcggcggcggcggctctgacatccagatgacccagtcccc
atctagcctgtctgccagcgtcggcgacagggtgaccatcacatgtcgcgcctctcaggatatcaggaactacctgaattggta
tcagcagaagcccggcaaggcccctaagctgctgatctactatacatccaggctggagtctggagtgccaagcaggttctccg
gatctggaagcggaaccgactacaccctgacaatctcctctctgcagcccgaggatttcgccacatactattgtcagcagggc
aataccctgccttggacatttggccagggcaccaaggtggagatcaagagctccggcggcggcggaagccagtccgtgctga
cccagcccccttctgtgagcggagcaccaggacagagggtgaccatcagctgcacaggcagctcctctaacatcggcgccgg
ctacgacgtgcactggtatcagcacctgccaggcacagcccccaagctgctgatctacggcaactctaatcggcctagcggcgt
gccagatcgcttctccggctctaagagcggcacctccgcctctctggccatcacaggcctgcaggccgaggacgagaccgatt
acttctgccagagctatgacagctccctgtccgcctgggtgtttggcggcggaaccaaggtgaccgtgctgggcggcggcggc
agcggcggcggcggcagcggcggcggcggctctcaggtgcagctggtgcagagcggcggcggcgtggtgcagcctggcg
gctccctgaggctgtcttgtgcagcaagcggattccccttttctagctatgcaatgtcctgggtgcgccaggcacctggcaaggg
actggagtgggtgagcgccatctccgccaatggcggaaccacatactatgccgactctgtgaagggcaggttcaccatctccag
agataactctaagaatacactgtacctgcagatgaactccctgagggccgaggacacagccgtgtactattgtgccaacaatggc
aattatagaggcgcctttgatatctggggccagggcaccatggtgacagtgtcctcttgataa
-G37-TRV-Fc
SEQ ID NO: 22
MDWTWILFLVAAATRVHSDKTHTCPPCPAPEAAGGPSVFLFPPKP
KDTLMISRTPEVTCVVVDVSHEAPEVKFNWYVDGVEVHNAKTKPCEEQYGSTY
RCVSVLTVLHQDWLNGKEYKCAVSNKALPAPIEKTISKAKGQPREPQVYTLPPSR
EEMTKNQVSLTCLVKGFYPSDIAVEWESNGQPENNYKTTPPVLDSDGSFFLYSKL
TVDKSRWQQGNVFSCSVMHEALHNHYTQKSLSLSPGKGGGGSGGGGSGGGGSG
GGGSGGGGSGGGGSDKTHTCPPCPAPEAAGGPSVFLFPPKPKDTLMISRTPEVTC
VVVDVSHEAPEVKFNWYVDGVEVHNAKTKPCEEQYGSTYRCVSVLTVLHQDW
LNGKEYKCAVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNV
FSCSVMHEALHNHYTQKSLSLSPGKGGGGSGGGGSQSVLTQPPSVSGAPGQRVTI
SCTGSSSNIGAGYDVHWYQHLPGTAPKLLIYGNSNRPSGVPDRFSGSKSGTSASL
AITGLQAEDETDYFCQSYDSSLSAWVFGGGTKVTVLGGGGSGGGGSGGGGSQV
QLVQSGGGVVQPGGSLRLSCAASGFPFSSYAMSWVRQAPGKGLEWVSAISANG
GTTYYADSVKGRFTISRDNSKNTLYLQMNSLRAEDTAVYYCANNGNYRGAFDI
WGQGTMVTVSSGGGGSEVQLVESGGGLVQPGGSLRLSCAASGYSFTGYTMNW
VRQAPGKGLEWVALINPYKGVSTYNQKFKDRFTISVDKSKNTAYLQMNSLRAED
TAVYYCARSGYYGDSDWYFDVWGQGTLVTVSSGGGGSGGGGSGGGGSDIQMT
QSPSSLSASVGDRVTITCRASQDIRNYLNWYQQKPGKAPKLLIYYTSRLESGVPSR
FSGSGSGTDYTLTISSLQPEDFATYYCQQGNTLPWTFGQGTKVEIKSSGGGGSQS
VLTQPPSVSGAPGQRVTISCTGSSSNIGAGYDVHWYQHLPGTAPKLLIYGNSNRP
SGVPDRFSGSKSGTSASLAITGLQAEDETDYFCQSYDSSLSAWVFGGGTKVTVLG
GGGSGGGGSGGGGSQVQLVQSGGGVVQPGGSLRLSCAASGFPFSSYAMSWVRQ
APGKGLEWVSAISANGGTTYYADSVKGRFTISRDNSKNTLYLQMNSLRAEDTAV
YYCANNGNYRGAFDIWGQGTMVTVSS
-G37-TRV-Fc with His tag
SEQ ID NO: 23
atggactggacctggatcctgttcctggtggctgccgccacaagggtgcacagcgataagacccacacat
gccctccctgtcctgcaccagaggcagccggcggaccttccgtgttcctgtttccacccaagccaaaggacaccctgatgatca
gcagaacccccgaggtgacatgcgtggtggtggacgtgtcccacgaggcacctgaggtgaagtttaactggtacgtggatggc
gtggaggtgcacaatgccaagacaaagccctgcgaggagcagtacggctctacctatagatgcgtgagcgtgctgacagtgct
gcaccaggattggctgaacggcaaggagtataagtgcgccgtgtctaataaggccctgcccgcccctatcgagaagaccatca
gcaaggcaaagggacagccaagggagcctcaggtgtacacactgcctccatctagagaggagatgaccaagaaccaggtga
gcctgacatgtctggtgaagggcttctatccatccgacatcgccgtggagtgggagtctaatggccagcccgagaacaattacaa
gaccacaccccctgtgctggactccgatggctcttttttctgtatagcaagctgaccgtggataagtccaggtggcagcagggc
aacgtgtttagctgctccgtgatgcacgaggccctgcacaatcactacacacagaagtctctgagcctgtcccctggcaagggc
ggcggcggcagcggcggcggcggcagcggcggcggcggcagcggcggcggcggcagcggcggcggcggcagcggc
ggcggcggctccgacaagacccacacatgcccaccctgtccagcccctgaggcagccggcggaccaagcgtgttcctgtttc
ctccaaagcctaaggataccctgatgatctcccggaccccagaagtcacctgcgtggtcgtggacgtgtctcacgaggcccccg
aggtgaagttcaactggtacgtggacggagtcgaagtgcacaatgccaagaccaagccatgtgaagagcagtacggctccacc
tatcgctgcgtgagcgtgctgacagtgctgcatcaggactggctgaatggcaaggaatataagtgcgccgtgagcaataaggcc
ctgcctgccccaatcgagaagaccatctctaaagccaaaggacagccaagggagccacaggtgtacacactgcccccttcccg
cgaagaaatgactaaaaaccaggtgtctctgacctgcctggtcaaaggcttctatcctagcgacatcgcagtggagtgggagtcc
aacggacagccagaaaataattacaagaccacaccacccgtgctggacagcgatggctccttctttctgtattctaaactgactgt
ggataagagcagatggcagcagggaaacgtgttttcttgtagcgtgatgcatgaggccctgcataaccactacacacagaagtc
cctgtctctgagtccaggcaagggcggcggcggcagcggcggcggcggctctcagagcgtgctgacacagcccccttccgt
gtctggagcaccaggacagcgggtgaccatcagctgcacaggcagctcctctaacatcggcgccggctacgacgtgcactgg
tatcagcacctgccaggaaccgcaccaaagctgctgatctacggcaactctaatagacctagcggcgtgccagatcggtttag
cggatccaagtctggcaccagcgcctccctggcaatcacaggactgcaggcagaggacgagacagattacttctgccagtcct
atgatagctccctgtctgcctgggtgtttggcggcggaaccaaggtgaccgtgctgggcggcggcggcagcggcggcggcgg
cagcggcggcggcggctcccaggtgcagctggtgcagagcggcggcggcgtggtgcagcctggcggctccctgagactgtct
tgtgccgccagcggcttccctttttctagctacgcaatgagctgggtgcggcaggcaccaggcaagggactggagtgggtgtcc
gccatctctgccaacggcggaaccacatactatgccgactccgtgaagggcaggttcaccatctccagagataactctaagaa
tacactgtacctgcagatgaacagcctgagggcagaggacaccgccgtgtactattgcgccaacaatggcaattataggggc
gcctttgatatctggggccagggcaccatggtgacagtgtccagcggcggcggcggctcggaggtgcagctggtggagagcg
gcggcggcctggtgcagccaggcggcagcctgaggctgtcctgtgcagcaagcggatactccttcaccggctatacaatgaat
tgggtgaggcaggcccctggcaagggcctggaatgggtggccctgatcaacccctacaagggcgtgtccacctataatcaga
agttcaaggaccgctttaccatctctgtggataagagcaagaacacagcctacctgcagatgaatagcctgagagccgagga
cacagccgtgtactattgcgcacggagcggatactatggcgactccgattggtattttgacgtgtggggccagggcaccctggt
gacagtgagcagcggcggcggcggcagcggcggcggcggcagcggcggcggcggctctgacatccagatgacccagtcccc
atctagcctgtctgccagcgtcggcgacagggtgaccatcacatgtcgcgcctctcaggatatcaggaactacctgaattggta
tcagcagaagcccggcaaggcccctaagctgctgatctactatacatccaggctggagtctggagtgccaagcaggttctccg
gatctggaagcggaaccgactacaccctgacaatctcctctctgcagcccgaggatttcgccacatactattgtcagcagggc
aataccctgccttggacatttggccagggcaccaaggtggagatcaagagctccggcggcggcggaagccagtccgtgctga
cccagcccccttctgtgagcggagcaccaggacagagggtgaccatcagctgcacaggcagctcctctaacatcggcgccgg
ctacgacgtgcactggtatcagcacctgccaggcacagcccccaagctgctgatctacggcaactctaatcggcctagcggcgt
gccagatcgcttctccggctctaagagcggcacctccgcctctctggccatcacaggcctgcaggccgaggacgagaccgatt
acttctgccagagctatgacagctccctgtccgcctgggtgtttggcggcggaaccaaggtgaccgtgctgggggcggcggc
agcggcggcggcggcagcggcggcggcggctctcaggtgcagctggtgcagagcggcggcggcgtggtgcagcctggcg
gctccctgaggctgtcttgtgcagcaagcggattccccttttctagctatgcaatgtcctgggtgcgccaggcacctggcaaggg
actggagtgggtgagcgccatctccgccaatggcggaaccacatactatgccgactctgtgaagggcaggttcaccatctccag
agataactctaagaatacactgtacctgcagatgaactccctgagggccgaggacacagccgtgtactattgtgccaacaatggc
aattatagaggcgcctttgatatctggggccagggcaccatggtgacagtgtcctctcaccaccaccaccaccactgataa
-G37-TRV-Fc with His tag
SEQ ID NO: 24
MDWTWILFLVAAATRVHSDKTHTCPPCPAPEAAGGPSVFLFPPKP
KDTLMISRTPEVTCVVVDVSHEAPEVKFNWYVDGVEVHNAKTKPCEEQYGSTY
RCVSVLTVLHQDWLNGKEYKCAVSNKALPAPIEKTISKAKGQPREPQVYTLPPSR
EEMTKNQVSLTCLVKGFYPSDIAVEWESNGQPENNYKTTPPVLDSDGSFFLYSKL
TVDKSRWQQGNVFSCSVMHEALHNHYTQKSLSLSPGKGGGGSGGGGSGGGGSG
GGGSGGGGSGGGGSDKTHTCPPCPAPEAAGGPSVFLFPPKPKDTLMISRTPEVTC
VVVDVSHEAPEVKFNWYVDGVEVHNAKTKPCEEQYGSTYRCVSVLTVLHQDW
LNGKEYKCAVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNV
FSCSVMHEALHNHYTQKSLSLSPGKGGGGSGGGGSQSVLTQPPSVSGAPGQRVTI
SCTGSSSNIGAGYDVHWYQHLPGTAPKLLIYGNSNRPSGVPDRFSGSKSGTSASL
AITGLQAEDETDYFCQSYDSSLSAWVFGGGTKVTVLGGGGSGGGGSGGGGSQV
QLVQSGGGVVQPGGSLRLSCAASGFPFSSYAMSWVRQAPGKGLEWVSAISANG
GTTYYADSVKGRFTISRDNSKNTLYLQMNSLRAEDTAVYYCANNGNYRGAFDI
WGQGTMVTVSSGGGGSEVQLVESGGGLVQPGGSLRLSCAASGYSFTGYTMNW
VRQAPGKGLEWVALINPYKGVSTYNQKFKDRFTISVDKSKNTAYLQMNSLRAED
TAVYYCARSGYYGDSDWYFDVWGQGTLVTVSSGGGGSGGGGSGGGGSDIQMT
QSPSSLSASVGDRVTITCRASQDIRNYLNWYQQKPGKAPKLLIYYTSRLESGVPSR
FSGSGSGTDYTLTISSLQPEDFATYYCQQGNTLPWTFGQGTKVEIKSSGGGGSQS
VLTQPPSVSGAPGQRVTISCTGSSSNIGAGYDVHWYQHLPGTAPKLLIYGNSNRP
SGVPDRFSGSKSGTSASLAITGLQAEDETDYFCQSYDSSLSAWVFGGGTKVTVLG
GGGSGGGGSGGGGSQVQLVQSGGGVVQPGGSLRLSCAASGFPFSSYAMSWVRQ
APGKGLEWVSAISANGGTTYYADSVKGRFTISRDNSKNTLYLQMNSLRAEDTAV
YYCANNGNYRGAFDIWGQGTMVTVSSHHHHHH
-G37-TRV-Fc+ (with mutations for half life extension)
SEQ ID NO: 25
atggactggacctggatcctgttcctggtggctgccgccacaagggtgcacagcgataagacccacacat
gccctccctgtcctgcaccagaggcagccggcggaccttccgtgttcctgtttccacccaagccaaaggacaccctgatgatca
gcagaacccccgaggtgacatgcgtggtggtggacgtgtcccacgaggcacctgaggtgaagtttaactggtacgtggatggc
gtggaggtgcacaatgccaagacaaagccctgcgaggagcagtacggctctacctatagatgcgtgagcgtgctgacagtgct
gcaccaggattggctgaacggcaaggagtataagtgcgccgtgtctaataaggccctgcccgcccctatcgagaagaccatca
gcaaggcaaagggacagccaagggagcctcaggtgtacacactgcctccatctagagaggagatgaccaagaaccaggtga
gcctgacatgtctggtgaagggcttctatccatccgacatcgccgtggagtgggagtctaatggccagcccgagaacaattacaa
gaccacaccccctgtgctggactccgatggctctttctttctgtatagcaagctgaccgtggataagtccaggtggcagcagggc
aacgtgtttagctgctccgtgctgcacgaggccctgcactcccactacacacagaagtctctgagcctgtcccctggcaagggc
ggcggcggcagcggcggcggcggcagcggcggcggcggcagcggcggcggcggcagcggcggcggcggcagcggc
ggcggcggctccgacaagacccacacatgcccaccctgtccagcccctgaggcagccggcggaccaagcgtgttcctgtttc
ctccaaagcctaaggataccctgatgatctcccggaccccagaagtcacctgcgtggtcgtggacgtgtctcacgaggcccccg
aggtgaagttcaactggtacgtggacggagtcgaagtgcacaatgccaagaccaagccatgtgaagagcagtacggctccacc
tatcgctgcgtgagcgtgctgacagtgctgcatcaggactggctgaatggcaaggaatataagtgcgccgtgagcaataaggcc
ctgcctgccccaatcgagaagaccatctctaaagccaaaggacagccaagggagccacaggtgtacacactgcccccttcccg
cgaagaaatgactaaaaaccaggtgtctctgacctgcctggtcaaaggcttctatcctagcgacatcgcagtggagtgggagtcc
aacggacagccagaaaataattacaagaccacaccacccgtgctggacagcgatggctccttctttctgtattctaaactgactgt
ggataagagcagatggcagcagggaaacgtgttttcttgtagcgtgctgcacgaggccctgcactcccactacacacagaagtc
cctgtctctgagtccaggcaagggcggcggcggcagcggcggcggcggctctcagagcgtgctgacacagcccccttccgt
gtctggagcaccaggacagcgggtgaccatcagctgcacaggcagctcctctaacatcggcgccggctacgacgtgcactgg
tatcagcacctgccaggaaccgcaccaaagctgctgatctacggcaactctaatagacctagcggcgtgccagatcggtttag
cggatccaagtctggcaccagcgcctccctggcaatcacaggactgcaggcagaggacgagacagattacttctgccagtcct
atgatagctccctgtctgcctgggtgtttggcggcggaaccaaggtgaccgtgctgggcggcggcggcagcggcggcggcgg
cagcggcggcggcggctcccaggtgcagctggtgcagagcggcggcggcgtggtgcagcctggcggctccctgagactgtct
tgtgccgccagcggcttccctttttctagctacgcaatgagctgggtgcggcaggcaccaggcaagggactggagtgggtgtcc
gccatctctgccaacggcggaaccacatactatgccgactccgtgaagggcaggttcaccatctccagagataactctaagaa
tacactgtacctgcagatgaacagcctgagggcagaggacaccgccgtgtactattgcgccaacaatggcaattataggggc
gcctttgatatctggggccagggcaccatggtgacagtgtccagcggcggcggcggctcggaggtgcagctggtggagagcg
gcggcggcctggtgcagccaggcggcagcctgaggctgtcctgtgcagcaagcggatactccttcaccggctatacaatgaat
tgggtgaggcaggcccctggcaagggcctggaatgggtggccctgatcaacccctacaagggcgtgtccacctataatcaga
agttcaaggaccgctttaccatctctgtggataagagcaagaacacagcctacctgcagatgaatagcctgagagccgagga
cacagccgtgtactattgcgcacggagcggatactatggcgactccgattggtattttgacgtgtggggccagggcaccctggt
gacagtgagcagcggcggcggcggcagcggcggcggcggcagcggcggcggcggctctgacatccagatgacccagtcccc
atctagcctgtctgccagcgtcggcgacagggtgaccatcacatgtcgcgcctctcaggatatcaggaactacctgaattggta
tcagcagaagcccggcaaggcccctaagctgctgatctactatacatccaggctggagtctggagtgccaagcaggttctccg
gatctggaagcggaaccgactacaccctgacaatctcctctctgcagcccgaggatttcgccacatactattgtcagcagggc
aataccctgccttggacatttggccagggcaccaaggtggagatcaagagctccggcggcggcggaagccagtccgtgctga
cccagcccccttctgtgagcggagcaccaggacagagggtgaccatcagctgcacaggcagctcctctaacatcggcgccgg
ctacgacgtgcactggtatcagcacctgccaggcacagcccccaagctgctgatctacggcaactctaatcggcctagcggcgt
gccagatcgcttctccggctctaagagcggcacctccgcctctctggccatcacaggcctgcaggccgaggacgagaccgatt
acttctgccagagctatgacagctccctgtccgcctgggtgtttggcggcggaaccaaggtgaccgtgctgggcggcggcggc
agcggcggcggcggcagcggcggcggcggctctcaggtgcagctggtgcagagcggcggcggcgtggtgcagcctggcg
gctccctgaggctgtcttgtgcagcaagcggattccccttttctagctatgcaatgtcctgggtgcgccaggcacctggcaaggg
actggagtgggtgagcgccatctccgccaatggcggaaccacatactatgccgactctgtgaagggcaggttcaccatctccag
agataactctaagaatacactgtacctgcagatgaactccctgagggccgaggacacagccgtgtactattgtgccaacaatggc
aattatagaggcgcctttgatatctggggccagggcaccatggtgacagtgtcctcttgataa
-G37-TRV-Fc+ (with mutations for half life extension)
SEQ ID NO: 26
MDWTWILFLVAAATRVHSDKTHTCPPCPAPEAAGGPSVFLFPPKPKDTLMISRTP
EVTCVVVDVSHEAPEVKFNWYVDGVEVHNAKTKPCEEQYGSTYRCVSVLTVLH
QDWLNGKEYKCAVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQ
QGNVFSCSVLHEALHSHYTQKSLSLSPGKGGGGSGGGGSGGGGSGGGGSGGGG
SGGGGSDKTHTCPPCPAPEAAGGPSVFLFPPKPKDTLMISRTPEVTCVVVDVSHE
APEVKFNWYVDGVEVHNAKTKPCEEQYGSTYRCVSVLTVLHQDWLNGKEYKC
AVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVLHEA
LHSHYTQKSLSLSPGKGGGGSGGGGSQSVLTQPPSVSGAPGQRVTISCTGSSSNIG
AGYDVHWYQHLPGTAPKLLIYGNSNRPSGVPDRFSGSKSGTSASLAITGLQAEDE
TDYFCQSYDSSLSAWVFGGGTKVTVLGGGGSGGGGSGGGGSQVQLVQSGGGV
VQPGGSLRLSCAASGFPFSSYAMSWVRQAPGKGLEWVSAISANGGTTYYADSVK
GRFTISRDNSKNTLYLQMNSLRAEDTAVYYCANNGNYRGAFDIWGQGTMVTVS
SGGGGSEVQLVESGGGLVQPGGSLRLSCAASGYSFTGYTMNWVRQAPGKGLEW
VALINPYKGVSTYNQKFKDRFTISVDKSKNTAYLQMNSLRAEDTAVYYCARSGY
YGDSDWYFDVWGQGTLVTVSSGGGGSGGGGSGGGGSDIQMTQSPSSLSASVGD
RVTITCRASQDIRNYLNWYQQKPGKAPKLLIYYTSRLESGVPSRFSGSGSGTDYT
LTISSLQPEDFATYYCQQGNTLPWTFGQGTKVEIKSSGGGGSQSVLTQPPSVSGAP
GQRVTISCTGSSSNIGAGYDVHWYQHLPGTAPKLLIYGNSNRPSGVPDRFSGSKS
GTSASLAITGLQAEDETDYFCQSYDSSLSAWVFGGGTKVTVLGGGGSGGGGSGG
GGSQVQLVQSGGGVVQPGGSLRLSCAASGFPFSSYAMSWVRQAPGKGLEWVSA
ISANGGTTYYADSVKGRFTISRDNSKNTLYLQMNSLRAEDTAVYYCANNGNYRG
AFDIWGQGTMVTVSS
-G37-TRV-Fc+ with His tag (with mutations for half life extension)
SEQ ID NO: 27
atggactggacctggatcctgttcctggtggctgccgccacaagggtgcacagcgataagacccacacat
gccctccctgtcctgcaccagaggcagccggcggaccttccgtgttcctgtttccacccaagccaaaggacaccctgatgatca
gcagaacccccgaggtgacatgcgtggtggtggacgtgtcccacgaggcacctgaggtgaagtttaactggtacgtggatggc
gtggaggtgcacaatgccaagacaaagccctgcgaggagcagtacggctctacctatagatgcgtgagcgtgctgacagtgct
gcaccaggattggctgaacggcaaggagtataagtgcgccgtgtctaataaggccctgcccgcccctatcgagaagaccatca
gcaaggcaaagggacagccaagggagcctcaggtgtacacactgcctccatctagagaggagatgaccaagaaccaggtga
gcctgacatgtctggtgaagggcttctatccatccgacatcgccgtggagtgggagtctaatggccagcccgagaacaattacaa
gaccacaccccctgtgctggactccgatggctctttctttctgtatagcaagctgaccgtggataagtccaggtggcagcagggc
aacgtgtttagctgctccgtgctgcacgaggccctgcactcccactacacacagaagtctctgagcctgtcccctggcaagggc
ggcggcggcagcggcggcggcggcagcggcggcggcggcagcggcggcggcggcagcggcggcggcggcagcggc
ggcggcggctccgacaagacccacacatgcccaccctgtccagcccctgaggcagccggcggaccaagcgtgttcctgtttc
ctccaaagcctaaggataccctgatgatctcccggaccccagaagtcacctgcgtggtcgtggacgtgtctcacgaggcccccg
aggtgaagttcaactggtacgtggacggagtcgaagtgcacaatgccaagaccaagccatgtgaagagcagtacggctccacc
tatcgctgcgtgagcgtgctgacagtgctgcatcaggactggctgaatggcaaggaatataagtgcgccgtgagcaataaggcc
ctgcctgccccaatcgagaagaccatctctaaagccaaaggacagccaagggagccacaggtgtacacactgcccccttcccg
cgaagaaatgactaaaaaccaggtgtctctgacctgcctggtcaaaggcttctatcctagcgacatcgcagtggagtgggagtcc
aacggacagccagaaaataattacaagaccacaccacccgtgctggacagcgatggctccttctttctgtattctaaactgactgt
ggataagagcagatggcagcagggaaacgtgttttcttgtagcgtgctgcacgaggccctgcactcccactacacacagaagtc
cctgtctctgagtccaggcaagggcggcggcggcagcggcggcggcggctctcagagcgtgctgacacagcccccttccgt
gtctggagcaccaggacagcgggtgaccatcagctgcacaggcagctcctctaacatcggcgccggctacgacgtgcactgg
tatcagcacctgccaggaaccgcaccaaagctgctgatctacggcaactctaatagacctagcggcgtgccagatcggtttag
cggatccaagtctggcaccagcgcctccctggcaatcacaggactgcaggcagaggacgagacagattacttctgccagtcct
atgatagctccctgtctgcctgggtgtttggcggcggaaccaaggtgaccgtgctgggcggcggcggcagcggcggcggcgg
cagcggcggcggcggctcccaggtgcagctggtgcagagcggcggcggcgtggtgcagcctggcggctccctgagactgtct
tgtgccgccagcggcttccctttttctagctacgcaatgagctgggtgcggcaggcaccaggcaagggactggagtgggtgtcc
gccatctctgccaacggcggaaccacatactatgccgactccgtgaagggcaggttcaccatctccagagataactctaagaa
tacactgtacctgcagatgaacagcctgagggcagaggacaccgccgtgtactattgcgccaacaatggcaattataggggc
gcctttgatatctggggccagggcaccatggtgacagtgtccagcggcggcggcggctcggaggtgcagctggtggagagcg
gcggcggcctggtgcagccaggcggcagcctgaggctgtcctgtgcagcaagcggatactccttcaccggctatacaatgaat
tgggtgaggcaggcccctggcaagggcctggaatgggtggccctgatcaacccctacaagggcgtgtccacctataatcaga
agttcaaggaccgctttaccatctctgtggataagagcaagaacacagcctacctgcagatgaatagcctgagagccgagga
cacagccgtgtactattgcgcacggagcggatactatggcgactccgattggtattttgacgtgtggggccagggcaccctggt
gacagtgagcagcggcggcggcggcagcggcggcggcggcagcggcggcggcggctctgacatccagatgacccagtcccc
atctagcctgtctgccagcgtcggcgacagggtgaccatcacatgtcgcgcctctcaggatatcaggaactacctgaattggta
tcagcagaagcccggcaaggcccctaagctgctgatctactatacatccaggctggagtctggagtgccaagcaggttctccg
gatctggaagcggaaccgactacaccctgacaatctcctctctgcagcccgaggatttcgccacatactattgtcagcagggc
aataccctgccttggacatttggccagggcaccaaggtggagatcaagagctccggcggcggcggaagccagtccgtgctga
cccagcccccttctgtgagcggagcaccaggacagagggtgaccatcagctgcacaggcagctcctctaacatcggcgccgg
ctacgacgtgcactggtatcagcacctgccaggcacagcccccaagctgctgatctacggcaactctaatcggcctagcggcgt
gccagatcgcttctccggctctaagagcggcacctccgcctctctggccatcacaggcctgcaggccgaggacgagaccgatt
acttctgccagagctatgacagctccctgtccgcctgggtgtttggcggcggaaccaaggtgaccgtgctgggcggcggcggc
agcggcggcggcggcagcggcggcggcggctctcaggtgcagctggtgcagagcggcggcggcgtggtgcagcctggcg
gctccctgaggctgtcttgtgcagcaagcggattccccttttctagctatgcaatgtcctgggtgcgccaggcacctggcaaggg
actggagtgggtgagcgccatctccgccaatggcggaaccacatactatgccgactctgtgaagggcaggttcaccatctccag
agataactctaagaatacactgtacctgcagatgaactccctgagggccgaggacacagccgtgtactattgtgccaacaatggc
aattatagaggcgcctttgatatctggggccagggcaccatggtgacagtgtcctctcaccaccaccaccaccactgataa
-G37-TRV-Fc+ with His tag (with mutations for half life extension)
SEQ ID NO: 28
MDWTWILFLVAAATRVHSDKTHTCPPCPAPEAAGGPSVFLFPPKP
KDTLMISRTPEVTCVVVDVSHEAPEVKFNWYVDGVEVHNAKTKPCEEQYGSTY
RCVSVLTVLHQDWLNGKEYKCAVSNKALPAPIEKTISKAKGQPREPQVYTLPPSR
EEMTKNQVSLTCLVKGFYPSDIAVEWESNGQPENNYKTTPPVLDSDGSFFLYSKL
TVDKSRWQQGNVFSCSVLHEALHSHYTQKSLSLSPGKGGGGSGGGGSGGGGSG
GGGSGGGGSGGGGSDKTHTCPPCPAPEAAGGPSVFLFPPKPKDTLMISRTPEVTC
VVVDVSHEAPEVKFNWYVDGVEVHNAKTKPCEEQYGSTYRCVSVLTVLHQDW
LNGKEYKCAVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNV
FSCSVLHEALHSHYTQKSLSLSPGKGGGGSGGGGSQSVLTQPPSVSGAPGQRVTI
SCTGSSSNIGAGYDVHWYQHLPGTAPKLLIYGNSNRPSGVPDRFSGSKSGTSASL
AITGLQAEDETDYFCQSYDSSLSAWVFGGGTKVTVLGGGGSGGGGSGGGGSQV
QLVQSGGGVVQPGGSLRLSCAASGFPFSSYAMSWVRQAPGKGLEWVSAISANG
GTTYYADSVKGRFTISRDNSKNTLYLQMNSLRAEDTAVYYCANNGNYRGAFDI
WGQGTMVTVSSGGGGSEVQLVESGGGLVQPGGSLRLSCAASGYSFTGYTMNW
VRQAPGKGLEWVALINPYKGVSTYNQKFKDRFTISVDKSKNTAYLQMNSLRAED
TAVYYCARSGYYGDSDWYFDVWGQGTLVTVSSGGGGSGGGGSGGGGSDIQMT
QSPSSLSASVGDRVTITCRASQDIRNYLNWYQQKPGKAPKLLIYYTSRLESGVPSR
FSGSGSGTDYTLTISSLQPEDFATYYCQQGNTLPWTFGQGTKVEIKSSGGGGSQS
VLTQPPSVSGAPGQRVTISCTGSSSNIGAGYDVHWYQHLPGTAPKLLIYGNSNRP
SGVPDRFSGSKSGTSASLAITGLQAEDETDYFCQSYDSSLSAWVFGGGTKVTVLG
GGGSGGGGSGGGGSQVQLVQSGGGVVQPGGSLRLSCAASGFPFSSYAMSWVRQ
APGKGLEWVSAISANGGTTYYADSVKGRFTISRDNSKNTLYLQMNSLRAEDTAV
YYCANNGNYRGAFDIWGQGTMVTVSSHHHHHH

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A nucleic acid molecule encoding one or more synthetic DNA encoded bispecific therapeutic antibodies, or a binding fragment thereof, wherein the one or more synthetic DNA encoded bispecific immune cell engager comprises at least one least one antigen binding domain specific for binding to carbonic anhydrase 9 (CA9), and at least one immune cell engaging domain.

2. The nucleic acid molecule of claim 1, wherein the immune cell engaging domain targets a cell selected from the group consisting of a T cell, an antigen presenting cell, a natural killer (NK) cell, a neutrophil and a macrophage.

3. The nucleic acid molecule of claim 1, wherein the immune cell engaging domain targets at least one T cell specific receptor molecule selected from the group consisting of CD3, the T cell receptor (TCR), CD28, CD16, NKG2D, Ox40, 4-1BB, CD2, CD5, CD40, FcgRs, FceRs, FcaRs and CD95.

4. The nucleic acid molecule of claim 3, wherein the immune cell engaging domain targets CD3.

5. The nucleic acid molecule of claim 1 comprising a nucleotide sequence encoding one or more sequences selected from the group consisting of:
a) an amino acid sequence having at least about 90% identity over an entire length of the amino acid sequence to an amino acid sequence selected from the group consisting of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, and SEQ ID NO:28;
b) a binding fragment of an amino acid sequence having at least about 90% identity over at least 65% of the amino acid sequence to an amino acid sequence selected from the group consisting of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, and SEQ ID NO:28;
c) an amino acid sequence selected from the group consisting of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, and SEQ ID NO:28; and
d) a binding fragment of an amino acid sequence comprising at least 65% of an amino acid sequence selected from the group consisting of SEQ ID NO:2, SEQ ID NO:4, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, and SEQ ID NO:28.

6. The nucleic acid molecule of claim 5, wherein the binding fragment comprises the six CDR sequences of the amino acid sequences of SEQ ID NO:2 and SEQ ID NO:4.

7. The nucleic acid molecule of claim 5, wherein the binding fragment comprises the twelve CDR sequences of the amino acid sequences of SEQ ID NO:2, SEQ ID NO:4, SEQ ID NO:6, and SEQ ID NO:8.

8. The nucleic acid molecule of claim 1, selected from the group consisting of:
a) a nucleotide sequence having at least about 90% identity over an entire length of the nucleic acid sequence to a nucleotide sequence selected from the group consisting of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27;
b) a fragment of a nucleotide sequence having at least about 90% identity over at least 65% of the nucleic acid sequence to a nucleotide sequence selected from the group consisting of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27;
c) a nucleotide sequence selected from the group consisting of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27; and
d) a fragment of a nucleotide sequence comprising at least 65% of a nucleotide sequence selected from the group consisting of SEQ ID NO:1, SEQ ID NO:3, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or SEQ ID NO:27.

9. The nucleic acid molecule of claim 8, wherein the fragment encodes the six CDR sequences of the amino acid sequences of SEQ ID NO:2 and SEQ ID NO:4.

10. The nucleic acid molecule of claim 8, wherein the fragment encodes the twelve CDR sequences of the amino acid sequences of SEQ ID NO:2, SEQ ID NO:4, SEQ ID NO:6, and SEQ ID NO:8.

11. The nucleic acid molecule of any one of claims 1-10, wherein the nucleotide sequence is operably linked to a nucleic acid sequence encoding an IgE leader sequence.

12. The nucleic acid molecule of any one of claims 1-11, wherein the nucleic acid molecule comprises an expression vector.

13. A composition comprising at least one nucleic acid molecule of any one of claims 1-12.

14. The composition of claim 13, further comprising a pharmaceutically acceptable excipient.

15. A method of preventing or treating a disease or disorder in a subject, the method comprising administering to the subject a nucleic acid molecule of any of claims 1-12 or a composition of any of claims 13-14.

16. The method of claim 15, wherein the disease is selected from the group consisting of a benign tumor, cancer and a cancer-associated disease.

17. The method of claim 16, wherein the disease is a cancer associated with CA9 expression.

18. The method of claim 17, wherein the disease is selected from the group consisting of renal cell carcinoma and hepatocellular carcinoma.


SEQUENCE LISTING (28 sequences)

SEQ ID NO: 1 - DNA, 333 bp, synthetic construct
  source 1..333: mol_type = other DNA
cagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctg

SEQ ID NO: 2 - protein, 111 aa, synthetic construct
QSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVL

SEQ ID NO: 3 - DNA, 357 bp, synthetic construct
  source 1..357: mol_type = other DNA
caggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagc

SEQ ID NO: 4 - protein, 119 aa, synthetic construct
QVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSS

SEQ ID NO: 5 - DNA, 366 bp, synthetic construct
  source 1..366: mol_type = other DNA
gaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagc

SEQ ID NO: 6 - protein, 122 aa, synthetic construct [sequence omitted]

SEQ ID NO: 7 - DNA, 327 bp, synthetic construct
  source 1..327: mol_type = other DNA
gacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcc

SEQ ID NO: 8 - protein, 109 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 9 - DNA, 1548 bp, synthetic construct
  source 1..1548: mol_type = other DNA
atggactggacctggatcctgttcctggtggctgccgccaccagagtgcactctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcctgataa

SEQ ID NO: 10 - protein, 514 aa, synthetic construct
MDWTWILFLVAAATRVHSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 11 - DNA, 1566 bp, synthetic construct
  source 1..1566: mol_type = other DNA
atggactggacctggatcctgttcctggtggctgccgccaccagagtgcactctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcccaccaccaccaccaccactgataa

SEQ ID NO: 12 - protein, 520 aa, synthetic construct
MDWTWILFLVAAATRVHSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 13 - DNA, 3030 bp, synthetic construct
  source 1..3030: mol_type = other DNA
atggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgatgcacgaggccctgcacaatcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgatgcatgaggccctgcataaccactacacacagaagtccctgtctctgagccctggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcctgataa

SEQ ID NO: 14 - protein, 1008 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 15 - DNA, 3048 bp, synthetic construct
  source 1..3048: mol_type = other DNA
atggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgatgcacgaggccctgcacaatcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgatgcatgaggccctgcataaccactacacacagaagtccctgtctctgagccctggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcccaccaccaccaccaccactgataa

SEQ ID NO: 16 - protein, 1014 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 17 - DNA, 3030 bp, synthetic construct
  source 1..3030: mol_type = other DNA
atggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgctgcacgaggccctgcactctcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgctgcacgaggccctgcactctcactacacacagaagtccctgtctctgagccctggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcctgataa

SEQ ID NO: 18 - protein, 1008 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 19 - DNA, 3048 bp, synthetic construct
  source 1..3048: mol_type = other DNA
atggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgctgcacgaggccctgcactctcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgctgcacgaggccctgcactctcactacacacagaagtccctgtctctgagccctggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcccaccaccaccaccaccactgataa

SEQ ID NO: 20 - protein, 1014 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 21 - DNA, 3780 bp, synthetic construct
  source 1..3780: mol_type = other DNA
atggactggacctggatcctgttcctggtggctgccgccacaagggtgcacagcgataagacccacacatgccctccctgtcctgcaccagaggcagccggcggaccttccgtgttcctgtttccacccaagccaaaggacaccctgatgatcagcagaacccccgaggtgacatgcgtggtggtggacgtgtcccacgaggcacctgaggtgaagtttaactggtacgtggatggcgtggaggtgcacaatgccaagacaaagccctgcgaggagcagtacggctctacctatagatgcgtgagcgtgctgacagtgctgcaccaggattggctgaacggcaaggagtataagtgcgccgtgtctaataaggccctgcccgcccctatcgagaagaccatcagcaaggcaaagggacagccaagggagcctcaggtgtacacactgcctccatctagagaggagatgaccaagaaccaggtgagcctgacatgtctggtgaagggcttctatccatccgacatcgccgtggagtgggagtctaatggccagcccgagaacaattacaagaccacaccccctgtgctggactccgatggctctttctttctgtatagcaagctgaccgtggataagtccaggtggcagcagggcaacgtgtttagctgctccgtgatgcacgaggccctgcacaatcactacacacagaagtctctgagcctgtcccctggcaagggcggcggcggcagcggcggcggcggcagcggcggcggcggcagcggcggcggcggcagcggcggcggcggcagcggcggcggcggctccgacaagacccacacatgcccaccctgtccagcccctgaggcagccggcggaccaagcgtgttcctgtttcctccaaagcctaaggataccctgatgatctcccggaccccagaagtcacctgcgtggtcgtggacgtgtctcacgaggcccccgaggtgaagttcaactggtacgtggacggagtcgaagtgcacaatgccaagaccaagccatgtgaagagcagtacggctccacctatcgctgcgtgagcgtgctgacagtgctgcatcaggactggctgaatggcaaggaatataagtgcgccgtgagcaataaggccctgcctgccccaatcgagaagaccatctctaaagccaaaggacagccaagggagccacaggtgtacacactgcccccttcccgcgaagaaatgactaaaaaccaggtgtctctgacctgcctggtcaaaggcttctatcctagcgacatcgcagtggagtgggagtccaacggacagccagaaaataattacaagaccacaccacccgtgctggacagcgatggctccttctttctgtattctaaactgactgtggataagagcagatggcagcagggaaacgtgttttcttgtagcgtgatgcatgaggccctgcataaccactacacacagaagtccctgtctctgagtccaggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctccggcggcggcggaagccagtccgtgctgacccagcccccttctgtgagcggagcaccaggacagagggtgaccatcagctgcacaggcagctcctctaacatcggcgccggctacgacgtgcactggtatcagcacctgccaggcacagcccccaagctgctgatctacggcaactctaatcggcctagcggcgtgccagatcgcttctccggctctaagagcggcacctccgcctctctggccatcacaggcctgcaggccgaggacgagaccgattacttctgccagagctatgacagctccctgtccgcctgggtgtttggcggcggaaccaaggtgaccgtgctgggcggcggcggcagcggcggcggcggcagcggcggcggcggctctcaggtgcagctggtgcagagcggcggcggcgtggtgcagcctggcggctccctgaggctgtcttgtgcagcaagcggattccccttttctagctatgcaatgtcctgggtgcgccaggcacctggcaagggactggagtgggtgagcgccatctccgccaatggcggaaccacatactatgccgactctgtgaagggcaggttcaccatctccagagataactctaagaatacactgtacctgcagatgaactccctgagggccgaggacacagccgtgtactattgtgccaacaatggcaattatagaggcgcctttgatatctggggccagggcaccatggtgacagtgtcctcttgataa

SEQ ID NO: 22 - protein, 1258 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSS

SEQ ID NO: 23 - DNA, 3798 bp, synthetic construct
  source 1..3798: mol_type = other DNA
atggactggacctggatcctgttcctggtggctgccgccacaagggtgcacagcgataagacccacacatgccctccctgtcctgcaccagaggcagccggcggaccttccgtgttcctgtttccacccaagccaaaggacaccctgatgatcagcagaacccccgaggtgacatgcgtggtggtggacgtgtcccacgaggcacctgaggtgaagtttaactggtacgtggatggcgtggaggtgcacaatgccaagacaaagccctgcgaggagcagtacggctctacctatagatgcgtgagcgtgctgacagtgctgcaccaggattggctgaacggcaaggagtataagtgcgccgtgtctaataaggccctgcccgcccctatcgagaagaccatcagcaaggcaaagggacagccaagggagcctcaggtgtacacactgcctccatctagagaggagatgaccaagaaccaggtgagcctgacatgtctggtgaagggcttctatccatccgacatcgccgtggagtgggagtctaatggccagcccgagaacaattacaagaccacaccccctgtgctggactccgatggctctttctttctgtatagcaagctgaccgtggataagtccaggtggcagcagggcaacgtgtttagctgctccgtgatgcacgaggccctgcacaatcactacacacagaagtctctgagcctgtcccctggcaagggcggcggcggcagcggcggcggcggcagcggcggcggcggcagcggcggcggcggcagcggcggcggcggcagcggcggcggcggctccgacaagacccacacatgcccaccctgtccagcccctgaggcagccggcggaccaagcgtgttcctgtttcctccaaagcctaaggataccctgatgatctcccggaccccagaagtcacctgcgtggtcgtggacgtgtctcacgaggcccccgaggtgaagttcaactggtacgtggacggagtcgaagtgcacaatgccaagaccaagccatgtgaagagcagtacggctccacctatcgctgcgtgagcgtgctgacagtgctgcatcaggactggctgaatggcaaggaatataagtgcgccgtgagcaataaggccctgcctgccccaatcgagaagaccatctctaaagccaaaggacagccaagggagccacaggtgtacacactgcccccttcccgcgaagaaatgactaaaaaccaggtgtctctgacctgcctggtcaaaggcttctatcctagcgacatcgcagtggagtgggagtccaacggacagccagaaaataattacaagaccacaccacccgtgctggacagcgatggctccttctttctgtattctaaactgactgtggataagagcagatggcagcagggaaacgtgttttcttgtagcgtgatgcatgaggccctgcataaccactacacacagaagtccctgtctctgagtccaggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctccggcggcggcggaagccagtccgtgctgacccagcccccttctgtgagcggagcaccaggacagagggtgaccatcagctgcacaggcagctcctctaacatcggcgccggctacgacgtgcactggtatcagcacctgccaggcacagcccccaagctgctgatctacggcaactctaatcggcctagcggcgtgccagatcgcttctccggctctaagagcggcacctccgcctctctggccatcacaggcctgcaggccgaggacgagaccgattacttctgccagagctatgacagctccctgtccgcctgggtgtttggcggcggaaccaaggtgaccgtgctgggcggcggcggcagcggcggcggcggcagcggcggcggcggctctcaggtgcagctggtgcagagcggcggcggcgtggtgcagcctggcggctccctgaggctgtcttgtgcagcaagcggattccccttttctagctatgcaatgtcctgggtgcgccaggcacctggcaagggactggagtgggtgagcgccatctccgccaatggcggaaccacatactatgccgactctgtgaagggcaggttcaccatctccagagataactctaagaatacactgtacctgcagatgaactccctgagggccgaggacacagccgtgtactattgtgccaacaatggcaattatagaggcgcctttgatatctggggccagggcaccatggtgacagtgtcctctcaccaccaccaccaccactgataa

SEQ ID NO: 24 - protein, 1264 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSHHHHHH

SEQ ID NO: 25 - DNA, 3780 bp, synthetic construct
  source 1..3780: mol_type = other DNA
atggactggacctggatcctgttcctggtggctgccgccacaagggtgcacagcgataagacccacacatgccctccctgtcctgcaccagaggcagccggcggaccttccgtgttcctgtttccacccaagccaaaggacaccctgatgatcagcagaacccccgaggtgacatgcgtggtggtggacgtgtcccacgaggcacctgaggtgaagtttaactggtacgtggatggcgtggaggtgcacaatgccaagacaaagccctgcgaggagcagtacggctctacctatagatgcgtgagcgtgctgacagtgctgcaccaggattggctgaacggcaaggagtataagtgcgccgtgtctaataaggccctgcccgcccctatcgagaagaccatcagcaaggcaaagggacagccaagggagcctcaggtgtacacactgcctccatctagagaggagatgaccaagaaccaggtgagcctgacatgtctggtgaagggcttctatccatccgacatcgccgtggagtgggagtctaatggccagcccgagaacaattacaagaccacaccccctgtgctggactccgatggctctttctttctgtatagcaagctgaccgtggataagtccaggtggcagcagggcaacgtgtttagctgctccgtgctgcacgaggccctgcactcccactacacacagaagtctctgagcctgtcccctggcaagggcggcggcggcagcggcggcggcggcagcggcggcggcggcagcggcggcggcggcagcggcggcggcggcagcggcggcggcggctccgacaagacccacacatgcccaccctgtccagcccctgaggcagccggcggaccaagcgtgttcctgtttcctccaaagcctaaggataccctgatgatctcccggaccccagaagtcacctgcgtggtcgtggacgtgtctcacgaggcccccgaggtgaagttcaactggtacgtggacggagtcgaagtgcacaatgccaagaccaagccatgtgaagagcagtacggctccacctatcgctgcgtgagcgtgctgacagtgctgcatcaggactggctgaatggcaaggaatataagtgcgccgtgagcaataaggccctgcctgccccaatcgagaagaccatctctaaagccaaaggacagccaagggagccacaggtgtacacactgcccccttcccgcgaagaaatgactaaaaaccaggtgtctctgacctgcctggtcaaaggcttctatcctagcgacatcgcagtggagtgggagtccaacggacagccagaaaataattacaagaccacaccacccgtgctggacagcgatggctccttctttctgtattctaaactgactgtggataagagcagatggcagcagggaaacgtgttttcttgtagcgtgctgcacgaggccctgcactcccactacacacagaagtccctgtctctgagtccaggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctccggcggcggcggaagccagtccgtgctgacccagcccccttctgtgagcggagcaccaggacagagggtgaccatcagctgcacaggcagctcctctaacatcggcgccggctacgacgtgcactggtatcagcacctgccaggcacagcccccaagctgctgatctacggcaactctaatcggcctagcggcgtgccagatcgcttctccggctctaagagcggcacctccgcctctctggccatcacaggcctgcaggccgaggacgagaccgattacttctgccagagctatgacagctccctgtccgcctgggtgtttggcggcggaaccaaggtgaccgtgctgggcggcggcggcagcggcggcggcggcagcggcggcggcggctctcaggtgcagctggtgcagagcggcggcggcgtggtgcagcctggcggctccctgaggctgtcttgtgcagcaagcggattccccttttctagctatgcaatgtcctgggtgcgccaggcacctggcaagggactggagtgggtgagcgccatctccgccaatggcggaaccacatactatgccgactctgtgaagggcaggttcaccatctccagagataactctaagaatacactgtacctgcagatgaactccctgagggccgaggacacagccgtgtactattgtgccaacaatggcaattatagaggcgcctttgatatctggggccagggcaccatggtgacagtgtcctcttgataa

SEQ ID NO: 26 - protein, 1258 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSS

SEQ ID NO: 27 - DNA, 3798 bp, synthetic construct
  source 1..3798: mol_type = other DNA
atggactggacctggatcctgttcctggtggctgccgccacaagggtgcacagcgataagacccacacatgccctccctgtcctgcaccagaggcagccggcggaccttccgtgttcctgtttccacccaagccaaaggacaccctgatgatcagcagaacccccgaggtgacatgcgtggtggtggacgtgtcccacgaggcacctgaggtgaagtttaactggtacgtggatggcgtggaggtgcacaatgccaagacaaagccctgcgaggagcagtacggctctacctatagatgcgtgagcgtgctgacagtgctgcaccaggattggctgaacggcaaggagtataagtgcgccgtgtctaataaggccctgcccgcccctatcgagaagaccatcagcaaggcaaagggacagccaagggagcctcaggtgtacacactgcctccatctagagaggagatgaccaagaaccaggtgagcctgacatgtctggtgaagggcttctatccatccgacatcgccgtggagtgggagtctaatggccagcccgagaacaattacaagaccacaccccctgtgctggactccgatggctctttctttctgtatagcaagctgaccgtggataagtccaggtggcagcagggcaacgtgtttagctgctccgtgctgcacgaggccctgcactcccactacacacagaagtctctgagcctgtcccctggcaagggcggcggcggcagcggcggcggcggcagcggcggcggcggcagcggcggcggcggcagcggcggcggcggcagcggcggcggcggctccgacaagacccacacatgcccaccctgtccagcccctgaggcagccggcggaccaagcgtgttcctgtttcctccaaagcctaaggataccctgatgatctcccggaccccagaagtcacctgcgtggtcgtggacgtgtctcacgaggcccccgaggtgaagttcaactggtacgtggacggagtcgaagtgcacaatgccaagaccaagccatgtgaagagcagtacggctccacctatcgctgcgtgagcgtgctgacagtgctgcatcaggactggctgaatggcaaggaatataagtgcgccgtgagcaataaggccctgcctgccccaatcgagaagaccatctctaaagccaaaggacagccaagggagccacaggtgtacacactgcccccttcccgcgaagaaatgactaaaaaccaggtgtctctgacctgcctggtcaaaggcttctatcctagcgacatcgcagtggagtgggagtccaacggacagccagaaaataattacaagaccacaccacccgtgctggacagcgatggctccttctttctgtattctaaactgactgtggataagagcagatggcagcagggaaacgtgttttcttgtagcgtgctgcacgaggccctgcactcccactacacacagaagtccctgtctctgagtccaggcaagggcggcggcggcagcggcggcggcggctctcagagcgtgctgacacagcccccttccgtgtctggagcaccaggacagcgggtgaccatcagctgcacaggcagctcctctaacatcggcgccggctacgacgtgcactggtatcagcacctgccaggaaccgcaccaaagctgctgatctacggcaactctaatagacctagcggcgtgccagatcggtttagcggatccaagtctggcaccagcgcctccctggcaatcacaggactgcaggcagaggacgagacagattacttctgccagtcctatgatagctccctgtctgcctgggtgtttggcggcggaaccaaggtgaccgtgctgggcggcggcggcagcggcggcggcggcagcggcggcggcggctcccaggtgcagctggtgcagagcggcggcggcgtggtgcagcctggcggctccctgagactgtcttgtgccgccagcggcttccctttttctagctacgcaatgagctgggtgcggcaggcaccaggcaagggactggagtgggtgtccgccatctctgccaacggcggaaccacatactatgccgactccgtgaagggcaggttcaccatctccagagataactctaagaatacactgtacctgcagatgaacagcctgagggcagaggacaccgccgtgtactattgcgccaacaatggcaattataggggcgcctttgatatctggggccagggcaccatggtgacagtgtccagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctccggcggcggcggaagccagtccgtgctgacccagcccccttctgtgagcggagcaccaggacagagggtgaccatcagctgcacaggcagctcctctaacatcggcgccggctacgacgtgcactggtatcagcacctgccaggcacagcccccaagctgctgatctacggcaactctaatcggcctagcggcgtgccagatcgcttctccggctctaagagcggcacctccgcctctctggccatcacaggcctgcaggccgaggacgagaccgattacttctgccagagctatgacagctccctgtccgcctgggtgtttggcggcggaaccaaggtgaccgtgctgggcggcggcggcagcggcggcggcggcagcggcggcggcggctctcaggtgcagctggtgcagagcggcggcggcgtggtgcagcctggcggctccctgaggctgtcttgtgcagcaagcggattccccttttctagctatgcaatgtcctgggtgcgccaggcacctggcaagggactggagtgggtgagcgccatctccgccaatggcggaaccacatactatgccgactctgtgaagggcaggttcaccatctccagagataactctaagaatacactgtacctgcagatgaactccctgagggccgaggacacagccgtgtactattgtgccaacaatggcaattatagaggcgcctttgatatctggggccagggcaccatggtgacagtgtcctctcaccaccaccaccaccactgataa

SEQ ID NO: 28 - protein, 1264 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSGGGGSQSVLTQPPSVSGAPGQRVTISCTGSSSNIGAGYDVHWYQHLPGTAPKLLIYGNSNRPSGVPDRFSGSKSGTSASLAITGLQAEDETDYFCQSYDSSLSAWVFGGGTKVTVLGGGGSGGGGSGGGGSQVQLVQSGGGVVQPGGSLRLSCAASGFPFSSYAMSWVRQAPGKGLEWVSAISANGGTTYYADSVKGRFTISRDNSKNTLYLQMNSLRAEDTAVYYCANNGNYRGAFDIWGQGTMVTVSSHHHHHH